SEQUENCE LISTING (7 sequences)

SEQ ID NO: 1 - protein, 8 aa, Artificial Sequence
  peptide
SIINFEKL

SEQ ID NO: 2 - protein, 127 aa, Artificial Sequence
  human BATF3 protein sequence; Q9NR55
MSQGLPAAGSVLQRSVAAPGNQPQPQPQQQSPEDDDRKVRRREKNRVAAQRSRKKQTQKADKLHEEYESLEQENTMLRREIGKLTEELKHLTEALKEHEKMCPLLLCPMNFVPVPPRPDPVAGCLPR

SEQ ID NO: 3 - protein, 118 aa, Artificial Sequence
  mouse BATF3 protein sequence; Q9D275
MSQGPPAVSVLQRSVDAPGNQPQSPKDDDRKVRRREKNRVAAQRSRKKQTQKADKLHEEHESLEQENSVLRREISKLKEELRHLSEVLKEHEKMCPLLLCPMNFVQLRSDPVASCLPR

SEQ ID NO: 4 - DNA, 97 bp, Artificial Sequence
  DNA encoding BCL2L11 shRNA(3)
tgctgttgacagtgagcgaaagcaaccttctgatgtaagatagtgaagccacagatgtatcttacatcagaaggttgcttgtgcctactgcctcgga

SEQ ID NO: 5 - DNA, 97 bp, Artificial Sequence
  DNA encoding BCL2L11 shRNA(1)
tgctgttgacagtgagcgcagcaaccttctgatgtaagtttagtgaagccacagatgtaaacttacatcagaaggttgctttgcctactgcctcgga

SEQ ID NO: 6 - DNA, 97 bp, Artificial Sequence
  DNA encoding BCL2L11 shRNA(2)
tgctgttgacagtgagcgaagttgtgacaagtcaacacaatagtgaagccacagatgtattgtgttgacttgtcacaactctgcctactgcctcgga

SEQ ID NO: 7 - RNA, 5098 nt, Artificial Sequence
  human BCL2L11 mRNA transcript variant 1; NM_138621.5
acuucgcuccgcgcagccgccuggucugcaguuuguuggagcucugcguccagcgccgcugccgcugccgccgccgccgccgccgccgccgccgccgccgccgccgccacuaccaccacuugauucuugcagccacccugcgaacccugccacacugcgaucgcaucaucgcgguauucgguucgcugcguucccgccgccaccgccucggcgcccuuucuuggcccuuguucccccaaaugucugacucugacucucggacugagaaacgcaagaaaaaaagaccaaauggcaaagcaaccuucugauguaaguucugagugugaccgagaagguagacaauugcagccugcggagaggccuccccagcucagaccuggggccccuaccucccuacagacagagccacaagguaauccugaaggcaaucacggaggugaaggggacagcugcccccacggcagcccucagggcccgcuggccccaccugccagcccuggcccuuuugcuaccagauccccgcuuuucaucuuuaugagaagauccucccugcugucucgauccuccaguggguauuucucuuuugacacagacaggagcccagcacccaugaguugugacaaaucaacacaaaccccaaguccuccuugccaggccuucaaccacuaucucagugcaauggcuuccaugaggcaggcugaaccugcagauaugcgcccagagauauggaucgcccaagaguugcggcguauuggagacgaguuuaacgcuuacuaugcaaggaggguauuuuugaauaauuaccaagcagccgaagaccacccacgaaugguuaucuuacgacuguuacguuacauuguccgccugguguggagaaugcauugacagguucuuugcggagccgagauaccaugcagacauuuugcuuguucaaaccaacaagacccagcaccgcggucuccuggugccauuauuaugcagccagcgguucucuuguggagggggcaggugacguuucagaagacaccgagcuggaugggacuaccuuucuguucaucaccacacagcagaauuucuaauggaaguuuguugugaauguaaaggagggagcauucuuugcuuuuuaauauacaaaccaugguuuuuuggagcaggauuuuguguaagaaugguguuuacaugcaguguguuuucccccucaccuucaauaagguuuuucaaaaaggaaauggaaacuuuuuaaccaauuugugaauaacuuuuguauuaaaauuuuaagaaccuacggccuauucucagaggauuauguaaccccugcaguggaaacugagccagcuaacuuaaaaagcugccuuaguuuauuuuuagagauuacagaauuuuuaaacagggagacgugugauauacucccucccuucccuacuauugccucucugaccuuuuuaaauuauuuuuaauaccaaaagaguucuuuugaaauggaacugauuaaaagggcagagggucuguugccagccugcauugauauaccagucccauuuguaaauauuuacguaccuuuauaaauucaguugcaucuguggcaaaauuucagacuauuuuugcgucuuuccucaucacuuuuugugaugcaacuccagucuggacucagaugcauagauuugguccaguguauuuucaugauaaagugaaauugagucagaacaagaguuaauaucugccuguaucuugcacaguucgagcgaucuguuauuaacugggaagcauuugguguugguuuucauuccauuucgacgagcauguuauugggaaguauucugaagaggcaauagcaguaauaacaacagacuuaagugcuacgccccuuugugcugcuggcuuuucugguugcaggcuuucccauggucacaggaugcacugucagcaucaggucccagagggccaccguguccauuacagcagaguccagcugcagcauccagcucacgcccucaugggaauuggcacaggccuggggcagggcuucugauggccauuugcuuggccuccugcauuuuaguccaacucacaguccacuagcuucacuccuuuaaauucacuuugaaacaggccucaucccacuuccaccagcaccauagaagaauaauucugggcagaagucuguuuuuuuucauuuuuccaggacaguuggauauugucaggccacuugugaccccagccauguagugagggugcucuuucucugugccugcuccuuaugagugcaguggaaggaagccacacacuggucagucauuucagaggcagcagaugcccagggagacccaagaaagagucagguuagggagcagugaaagugaggagggaagacaauucugugaacucuguaacucuuaaaauuuuugaaaacuccaucguuaaacaacuuuuaaaagaaauaacuaaauuuucaaaugaguaagcagugccaccaacuaguguuuugcccgauagaagagccagcauguucacguuauuuaaauuagguggaaaaaucuaaacauuuuuaucuucauaauuuaaaaaauauauauguauauauugcauauucacuuuuuccuuuagguagagaugauuucaauccaaauacucuuacuuuaaaaaauuuccuuuccccaagaaucuccuugggacuuugacuuauuuuuaaagcuguguuggagcucaucuuguucccugaugugucucgagcccauugguagggucauacaaagcccacgguuacaagcagugguaggauugcagccgugggccugcuggacacacacauacaccaaagauguauuuggaucugggcacccccucccaggaucccuguacucacgugccagucuccugacuagagcacuuuacucuguuuccucagcccugcagccccugggagcacacacugggugcagcccugggccaggcacgggaggcccugcccugugcugcccaggggcugugugcaccacaugagcacauuucccucuggccuggcggccuccaggcuggcuguggaaacaguuccugaggaaauuagagauucuaugaauuguaggaguauuaaagaccaggcuguuggcaccagaacuuaaagcgaugacuggaugucucuguacuguauguaucugguuaucaagaugccucugugcagaaaguaugccucccguggguauacguuuuuaccuuuuuuaaaaaacauuuuuguagaaaaaauaauuaaauccccuuuuuggaaacuuacugcagguuuugugccuugacaaccucucccuaugugagguuuguaaaaaguguccugugacuuaacacagaaacgcaauaaacacacacaaaauaguuucaugagugauucuucagaugcccuucccaacugguuaguugaucaagaauuuuggggguggggguugcggagaaaucaaguuuaaaauuccuucugauuaaaaaaauauaguggaauacaauugucugccguuuccccuucuuaauguauauauugugaguauuuauuagauucguaggucauauuacuuaucaacugagccaaaugucugugugcaauuguguuuccuuuaccuuguaaaauuuuguacagcauaaauaaguaaaaaaaucacuguuuuucucaacuuuuucaaaaucaaggauuguaaauauuguagauucuuuuucugugugauguguccuacuguuucauaaugcuguaacuuguagaaauauuguauauuuauuuucugcuuauuuaaugucuuaauuucugaaaaguauuaacaucccugucucccacuccccugccgucccaugaaguuaacuccugagaguugucgggggugacuggagagcucauugcagaccacgugguccuccaggguggcucuccaccuucggguccugguauuuccagucaaguggguuucaauucuugggcuuugccgcccuuaugaugaaguguguguuugaugccagugagaaacucagucuggcaggcuacaaaauucuacuccaagaaauacccagcaaccuucuguuuguuccaaagcaacuagcuuaucaugcaagcaaauuuugcugacuccaggcuuuaucuuuaggaaaacaaaaaaaccaaaguauuaucagcaggugggaaagauuuuucuauugaaaauuuaucccugacaacucagcguuuagaaaagaaauaaaaugugccacuuccagaggugcugcauugcaguuguucagggcuagggccaggcaggacaagugaaugggugggacagguggcuccugccuaaggaccaccucaggccacuaaccccuuguggacaacugugaguagcuggguuuucccccaccugcugugcaacuuccugugcuuugagguuggacuaacuugucuucaggagcuaauuaacuguacagcccuccccacgccccacccauacggucacugcauuuggucagccugcuucuucaggucgaugcccuccuucugauacuccaucuccuucaggggagguuggggccccacuggacugggugucaagaugugaaagcuuaugggagcuuuaaggagacuucauggugguuccaugcaggugguucugccaucccugcugauuuagccuggugccuguguguguccacucacguacacguggggugggggaaacgugucuacagaugacgcuaaaucaguuggggucuacucuaaacagcauuguguguaagaagcauccucaagcucccaguuaaguaacuugacuacuuuuauuugggaauuucagacuauagaagcucucuuauguuuuauguccagauucugugaccacuaguuacuguaucagaacucaucagguacccacuuauaaauagcacugaucuggcuguauacugauccaucacuaaccuguuuucuaggacccagcguauguagcauuuguauugcaguuucccuggcuuacuuguguuuugcacugaugaauuuugacaggguaauugccacuuuacuugugcaauacugcuguaaauaacugcagauuuuuaaacaaucuuuuauguuaauuuuauaaaaauaaaacuuucaacuagu

(19) United States
(12) Patent Application Publication
(10) Pub. No.: US 2023/0303975 A1
(43) Pub. Date: Sep. 28, 2023

(54) MODIFIED LYMPHOCYTES

(71) Applicant: JULIUS-MAXIMILIANS-UNIVERSITÄT WÜRZBURG, Würzburg (DE)

(72) Inventors: Wolfgang KASTENMÜLLER, Würzburg (DE); Marco Antonio ATAIDE SILVA, Sao Paulo (BR); Michael HUDECEK, Höchberg (DE)

(21) Appl. No.: 18/021,387

(22) PCT Filed: Aug. 20, 2021
(86) PCT No.: PCT/EP2021/073143

§ 371 Date: Feb. 14, 2023

(30) Foreign Application Priority Data
Aug. 21, 2020 (EP) 20192108.7

(51) Int. Cl. C12N 5/0783; C12N 15/113; A61P 35/00

(52) U.S. Cl. CPC C12N 5/0636; C12N 15/1135; A61P 35/00; C12N 2510/00; C12N 2310/14

(57) ABSTRACT

The present invention relates to a lymphocyte, modified to exhibit a reduced BCL2L11 level and/or an increased BATF3 level. The invention also relates to a method for producing such lymphocytes and a pharmaceutical composition comprising such lymphocytes. The lymphocytes and the pharmaceutical composition of the present invention may be used in methods for treating a disease in a patient.

### FIELD OF INVENTION

The present invention relates to a lymphocyte, modified to exhibit a reduced BCL2L11 level and/or an increased BATF3 level. The invention also relates to a method for producing such lymphocytes and a pharmaceutical composition comprising such lymphocytes. The lymphocytes and the pharmaceutical composition of the present invention may be used in methods for treating a disease in a patient.

### BACKGROUND

Upon encounter with dendritic cells in the context of infections, naïve antigen-specific CD8 T cells undergo activation (priming) and consecutively a massive expansion in order to generate the required effector to target ratio to effectively eliminate infected cells in the body. At the peak of the response, expanded CD8 T cells comprise a spectrum of differentiation states ranging from short-lived effector cells to long-lived memory cells [1]. Importantly, the quality of the developing memory T cell response is already determined during the first days of activation. One critical element for the optimal priming and programming of memory CD8 T cells are helper signals provided by cognate CD4 T cells [2]. The inventors and others have previously shown that a subset of conventional dendritic cells (cDC1) are the critical cellular platform on which these helper signals are exchanged [3, 4]. Currently, it is still unclear which transcriptional programs are required to promote their longevity and capacity to mount effective recall responses. Revealing these programs and its transcriptional elements is critical because they underlie CD8 memory stemness or in other words the capacity for engraftment, self-renewal and multipotency [5, 6, 7]. These in turn are essential properties of CD8 T cells that ultimately determine the efficacy of successful immunotherapy in humans.

The family of AP-1 (activator protein-1) transcription factors comprises numerous basic region-leucine zipper (bZIP) proteins that belong to different families (JUN, FOS, ATF, BATF, MAF) and form heterodimers to execute transcriptional activity. AP-1 transcription factors play a central role in every tissue and cell type and regulate cell death, survival and proliferation [8]. The overall regulation of AP-1 activity and its molecular elements is highly complex and involves transcriptional coactivators, modulation by MAP kinases and transcriptional inhibitors such as the BATF (basic leucine zipper transcriptional factor ATF-like) family of proteins [9]. BATF proteins play important roles in the development and function of both myeloid and lymphoid cell populations [10]. They execute these functions not only as a part and negative regulator of the AP-1 complex, but also promote transcriptional activity by interacting with IRF (interferon regulatory factor) family members [11]. BATF3 was first identified in human T cells and was later found to play a critical role for the development of the cDC1 subset of conventional dendritic cells [12, 13, 14, 15]. BATF3 is expressed across all conventional DC subsets; however, it appears to have only a critical function within cDC1. Batf3-deficient animals lack CD103-expressing migratory DC and a varying fraction of splenic CD8α DCs dependent on the genetic background (129 vs C57BL/6), the commensal communities and the infection history [16]. Thus, Batf3-deficient mice became a key model for elucidating the in vivo functions of cDC1. Other members of the BATF family, namely BATF and BATF2, appear to be dispensable for DC development; however, they can cross-compensate some functions of BATF3 based on the homology of the leucine-zipper domain between BATF proteins [17].

In murine lymphoid cells, Batf rather than Batf3 plays a central role for their function, development and differentiation. In particular, BATF regulates various CD4 T helper subsets like Th17, Tfh and Tr1 cells [18, 19, 20, 21]. In cytotoxic CD8 T cells, BATF was shown to be critical for T cell effector differentiation. During chronic infections BATF is required for expansion and differentiation, yet it also drives exhaustion and therefore inhibits CD8 T cell effector functionality [22, 23, 24]. Mechanistically, BATF functions within the AP-1 complex, interacts with cJUN and executes transcriptional activity together with IRF4 leading to reduced anabolic metabolism and a lowered mitochondrial membrane potential. Thereby, BATF regulates a large network of key transcription factors for CD8 T cell differentiation, epigenetic modification, T cell metabolism and survival [25, 26].

Upon chronic antigen exposure or chronic activation, lymphocytes such as T cells progressively reach a state with highly impaired effector function (e.g. killing capacity and cytokine production) and capacity to proliferate. This condition has been termed lymphocyte or T cell exhaustion and can even result in the physical depletion of responding (therapeutic) cells. As a result, the therapeutic effect of modified lymphocytes can be absent, suboptimal, and/or decline over time.

There is thus still a need for new means for sustained immunotherapy using modified lymphocytes.

### DESCRIPTION OF INVENTION

The present invention aims to overcome unmet clinical needs by providing improved lymphocytes for therapeutic treatment of patients.

In the current study the inventors identified a specific and lymphocyte intrinsic role for BATF3. In contrast to Batf-deficiency, loss of Batf3 did not impact CD8 T cell differentiation or their capacity to produce inflammatory cytokines. Instead, Batf3-deficient CD8 T cells showed a specific defect in T cell memory formation following, e.g., viral or bacterial infections, based on dysregulated expression of the proapoptotic factor BCL2L11 (BIM). Vice versa, overexpression of BATF3 augmented CD8 T cell fitness and promoted their persistence and memory development. Mechanistically, BATF3 altered the expression pattern of costimulatory and inhibitory receptors on CD8 T cells, optimized their metabolism and inhibited cell death via suppression of BCL2L11. These concrete findings on CD8 T cells plausibly apply to other lymphocytes because Batf3-deficient NK cells also show an increased contraction as compared to WT NK cells. Lymphocyte contraction refers to a phase after population expansion that is characterized by a dramatic (90-95%) loss of effector cells are that are rapidly eliminated by apoptosis. Additionally, BCL2L11 has been implicated in the survival of ILC (innate lymphocytes) like NK cells (Reference: PMID: 24958849).

By increasing the lifespan of lymphocytes, such as murine and human CD8 T cells and NK cells, BATF3 and its target BCL2L11, are promising candidates to enhance adoptive immunotherapy against cancer and infections.

Interestingly, another study by Lynn et al., 2019 [45] describes BATF3 as a factor that promotes exhaustion of CAR T cells and thus suggests that down-regulation, rather than up-regulation of BATF3 is desirable in CAR T cells in order to reduce T cell exhaustion. Lynn et al. have only indirectly tested this notion by overexpression of cJUN in order to outcompete possible negative effects of BATF and BATF3. However, the present inventors surprisingly found that increasing the level of BATF3 or reducing the levels of its key target BIM (BCL2L11) can increase the in vivo survival of lymphocytes, and especially T cells. Putting the study by Lynn et al., 2019 and the inventors' studies together, the inventors conclude that, surprisingly, both reduced and increased levels of BATF3 can enhance the in vivo activity, e.g. the anti-tumor activity of CD8 T cells, depending on the context and the abundance of transcriptional binding partners.

Accordingly, the present invention provides the following preferred embodiments:
[1] Lymphocyte, modified to exhibit a reduced Bcl-2-like protein 11 (BCL2L11) level and/or increased BATF3 level.
[2] Lymphocyte according to [1] that is modified to exhibit a reduced BCL2L11 level.
[3] Lymphocyte according to any one of the preceding items, wherein the BCL2L11 level is a BCL2L11 protein activity level and/or a BCL2L11 protein quantity level, preferably a BCL2L11 protein quantity level.
[4] Lymphocyte according to any one of the preceding items, comprising an exogenous inhibitor of BCL2L11 protein activity in the lymphocyte.
[5] Lymphocyte according to any one of the preceding items, comprising an exogenous activator of BCL2L11 protein degradation in the lymphocyte.
[6] Lymphocyte according to any one of the preceding items, wherein the BCL2L11 levels is a BCL2L11 expression level.
[7] Lymphocyte according to any one of the preceding items, comprising an exogenous inhibitor of BCL2L11 expression in the lymphocyte.
[8] Lymphocyte according to any one of the preceding items, comprising an exogenous inhibitor of transcription from an endogenous BCL2L11 gene and/or an inhibitor of translation of an endogenous BCL2L11 mRNA.
[9] Lymphocyte according to any one of the preceding items, comprising an exogenous RNAi effector molecule for reducing BCL2L11 expression in the lymphocyte.
[10] Lymphocyte according to item [9], wherein the RNAi effector molecule is an siRNA or an shRNA for reducing BCL2L11 expression in the lymphocyte.
[11] Lymphocyte according to any one of the preceding items, comprising an expression cassette for an siRNA or an shRNA for reducing BCL2L11 expression in the lymphocyte, wherein the expression cassette optionally has been integrated into the genome of lymphocyte.
[12] Lymphocyte according to any one of the preceding items, in which an endogenous BCL2L11 gene has been genetically modified in order to reduce or disrupt BCL2L11 expression, wherein optionally the endogenous BCL2L11 gene has been genetically modified in its promoter region.
[13] Lymphocyte according to any one of the preceding items that is modified to exhibit an increased BATF3 level, preferably human or murine BATF3 level.
[14] Lymphocyte according to any one of the preceding items, wherein the BATF3 level is a BATF3 protein activity level and/or a BATF3 protein quantity level, preferably a BATF3 protein quantity level.
[15] Lymphocyte according to any one of the preceding items, comprising an activator of BATF3 protein activity in the lymphocyte.
[16] Lymphocyte according to any one of the preceding items, comprising an exogenous inhibitor of BATF3 protein degradation in the lymphocyte.
[17] Lymphocyte according to any one of the preceding items, wherein the BATF3 level is a BATF3 expression level in the lymphocyte.
[18] Lymphocyte according to any one of the preceding items, comprising an exogenous activator of BATF3 expression in the lymphocyte.
[19] Lymphocyte according to any one of the preceding items, comprising an exogenous nucleic acid molecule for overexpressing BATF3, optionally human BATF3, wherein the nucleic acid molecule optionally has been integrated into the genome of the lymphocyte.
[20] Lymphocyte according to any one of the preceding items, comprising an exogenous activator of transcription from an endogenous BATF3 gene and/or activator of translation of an endogenous BATF3 mRNA.
[21] Lymphocyte according to any one of the preceding items, in which an endogenous BATF3 gene has been genetically modified in order to increase BATF3 expression, wherein optionally the endogenous BATF3 gene has been genetically modified in its promoter region.
[22] Lymphocyte,
   a) comprising an exogenous inhibitor of BCL2L11 protein activity in the lymphocyte; or
   b) comprising an exogenous activator of BCL2L11 protein degradation; or
   c) comprising an exogenous inhibitor of BCL2L11 expression in the lymphocyte and/or an (exogenous) expression cassette for expressing an inhibitor of BCL2L11 expression in the lymphocyte;
   or
   d) in which an endogenous BCL2L11 gene has been genetically modified in order to reduce or disrupt BCL2L11 expression, wherein optionally the endogenous BCL2L11 gene has been genetically modified in its promoter region.
[23] Lymphocyte,
   a) comprising an exogenous activator of BATF3 protein activity in the lymphocyte; or
   b) comprising an exogenous inhibitor of BATF3 protein degradation; or
   c) comprising an exogenous activator of BATF3 expression in the lymphocyte and/or a (exogenous) nucleic acid molecule for overexpressing BATF3 in the lymphocyte; or
   d) in which an endogenous BATF3 gene has been genetically modified in order to increase BATF3 expression, wherein optionally the endogenous BATF3 gene has been genetically modified in its promoter region.
[24] Method for producing a lymphocyte that exhibits a reduced BCL2L11 level and/or increased BATF3 level, wherein the method comprises
   a) introducing into the lymphocyte an exogenous inhibitor of BCL2L11 protein activity in the lymphocyte; or
   b) introducing into the lymphocyte an exogenous activator of BCL2L11 protein degradation in the lymphocyte; or
   c) introducing into the lymphocyte an exogenous inhibitor of BCL2L11 expression in the lymphocyte or an expression cassette for expressing an inhibitor of BCL2L11 expression in the lymphocyte; or
   d) genetically modifying an endogenous BCL2L11 gene in order to reduce or disrupt BCL2L11 expression, wherein optionally the endogenous BCL2L11 gene has been genetically modified in its promoter region; or
   e) introducing into the lymphocyte an exogenous activator of BATF3 protein activity in the lymphocyte; or
   f) introducing into the lymphocyte an exogenous inhibitor of BATF3 protein degradation; or
   g) introducing into the lymphocyte an exogenous activator of BATF3 expression and/or a (exogenous) nucleic acid molecule for overexpressing BATF3 in the lymphocyte; or
   h) genetically modifying an endogenous BATF3 gene in the lymphocyte in order to increase BATF3 expression, wherein optionally the endogenous BATF3 gene has been genetically modified in its promoter region.
[25] Lymphocyte obtainable by the method according to [24].
[26] Lymphocyte according to any one of [1]-[23] or [25], or method according to [24], wherein the lymphocyte is a mammalian lymphocyte.
[27] Lymphocyte according to any one of [1]-[23] or [25], or method according to [24], wherein the lymphocyte is a human lymphocyte.
[28] Lymphocyte according to any one of [1]-[23] or [25], or method according to [24], or lymphocyte or method according to any one of [26]-[27], wherein the lymphocyte is a T cell or an innate lymphoid cell, such as an ILC1, ILC2, ILC3 or NK cell.
[29] Lymphocyte according to any one of [1]-[23] or [25], or method according to [24], or lymphocyte or method according to any one of [26]-[28], wherein the lymphocyte is a T cell, such as a CD8+ T cell or a CD4+ T cell.
[30] Lymphocyte according to any one of [1]-[23] or [25], or method according to [24], or lymphocyte or method according to any one of [26]-[29], wherein the lymphocyte is a CD8+ T cell.
[31] Lymphocyte according to any one of [1]-[23] or [25], or method according to [24], or lymphocyte or method according to any one of [26]-[30], wherein the lymphocyte is a cell expressing a chimeric antigen receptor (CAR).
[32] Lymphocyte according to any one of [1]-[23] or [25], or method according to [24], or lymphocyte or method according to any one of [26]-[31], wherein the lymphocyte is a T cell expressing a chimeric antigen receptor (CAR).
[33] Lymphocyte according to any one of [1]-[23] or [25], or method according to [24], or lymphocyte or method according to any one of [26]-[32], wherein the lymphocyte exhibits increased survival in a (human) subject, for example after administration to the subject.
[34] Lymphocyte according to any one of [1]-[23] or [25], or method according to [24], or lymphocyte or method according to any one of [26]-[33], wherein the lymphocyte exhibits increased expression of IL7R in a (human) subject, for example after administration to the subject.
[35] Composition comprising a lymphocyte according to any one of [1]-[23] or [25]-[34], wherein the composition is optionally a pharmaceutical composition.
[36] Lymphocyte according to any one of [1]-[23] or [25]-[34], or composition according to [35] for use in medicine.
[37] Lymphocyte according to any one of [1]-[23] or [25]-[34], or composition according to [35] for use in treating cancer, an infectious disease, or an autoimmune disease, such as a chronic inflammatory disease or degenerative disease.
[38] Lymphocyte for use according to [36] or [37], wherein the lymphocyte exhibits an improved therapeutic activity and/or therapeutic outcome.

### BRIEF DESCRIPTION OF THE DRAWINGS

FIG. 1. BATF3 has a CD8 T cell intrinsic function.

(a, b) Wildtype (WT) C57BL/6 and Batf3^{-/-} mice were infected with Vaccinia Virus (VV). Antigen-specific (B8R) CD8⁺ T cells were analyzed on day 8 post primary infection (prime) and day 6 post secondary infection (recall). (c) Batf3 expression was assessed via qRT-PCR in CD8 T cells at different time points after in vitro anti-CD3/CD28 activation. (d, e) Quantification of antigen-specific (B8R) CD8⁺ T cells after prime (d8; VV) and recall response (d6; VV) of bone marrow chimeric chimeric mice. (f) Representative dotplots and (g) analysis of the relative abundance of OT-I T cells (WT and Batf3^{-/-}) after heterologous (VV-OVA or L.m.-OVA) primary and secondary infections. (h, i) OT-I T cells were isolated after prime or prime plus recall and restimulated with SIINFEKL (1 μg/ml, SEQ ID NO: 1) for 4 hours. Cytokine production was evaluated by FACS. (j) OT-I state of differentiation was assessed at d8 post primary VV-OVA infection. Data are representative of two (a, b; n=3), (c; n=2×3 technical replicates), (d, e; n=6 at prime and n=7 at recall), or three independent experiments (f-i; n=5), (j; n=4). Error bars indicate the mean±SEM. Comparison between groups was calculated using the two-tailed unpaired Student's t-test. **=p<0.01; ***=p<0.001.

FIG. 2. BATF3 regulates memory transition and survival of CD8 T cells.

(a, b) Kinetic analysis of cotransferred WT and Batf3^{-/-} OT-I cells in the blood and spleen of WT recipients after VV-OVA i.p. infection. (c) Representative dotplots and (d) statistical analysis of memory CD8 T cell subsets from WT and Batf3^{-/-} OT-I cells on d30 post infection. (e) 2-photon image of the mouse ear showing WT (tdTomato) and Batf3^{-/-} (GFP) OT-I cells d30 post infection. Collagen is represented by the second harmonic generation signal (SHG). Scale bar=70 μm. (f) Representative dotplots and statistical analysis of WT and Batf3^{-/-} OT-I cells extracted from mouse ear skin. (g) Memory populations (WT and Batf3^{-/-}) were re-isolated and re-transferred at a 50/50 ratio into new WT recipients and analysed on day 6 post VV-OVA infection. Data are representative of two (c, d n=3; f n=6) or three (a, b; n=4, g; n=6) independent experiments. Error bars indicate the mean±SEM. Comparison between groups was calculated using the (a, b) One sample t-test. ***=p=0.0003; (d, g) two-tailed paired Student's t-test. *=p≤0.05; **=p<0.01.

FIG. 3. The lack of BATF3 impacts on CD8 T cell fitness.

(a-c) Cells were isolated from spleens at different days post-VV-OVA infection. (a) Representative dotplots and (b) statistical analysis of the relative abundance of cotransferred WT (GFP) and Batf3^{-/-} (CD45.1) OT-I cells. (c) Representative dot plots and analysis of Ki-67 staining within CX3CR1⁺ WT (GFP) and Batf3^{-/-} (CD45.1) OT-I cells. (d, e) Cells were analysed at (d) d8 and (e) d12 post-VV-OVA infection. Representative dotplots, histograms and statistical analysis of mitochondrial membrane potential of the cotransferred WT (GFP) and Batf3^{-/-} (CD45.1) OT-I cells. Data are representative of two (a, b; n=4) or three (c-e; n=4) independent experiments. Error bars indicate the mean±SEM. Comparison between groups was calculated using the two-tailed paired Student's t-test. *=p≤0.05; **=p<0.01.

FIG. 4. BATF3 regulates CD8 T cell survival via the BCL2L11

(a, b) RT-PCR of Batf3, Pfr1 and Batf from ex vivo isolated cotransferred WT and Batf3^{-/-} OT-I cells after VV infection. (c) Significant DE genes comparing WT and Batf3^{-/-} CD8 T cells (48 h and 72 h post activation, Extended Data 4b) were subjected to pathway analysis. Top 10 significant pathways are displayed with corresponding p-value and combined enrichment score. (d, e) Representative histograms and statistical analysis of BCL2L11 protein levels on WT and Batf3^{-/-} OT-I cells (d) after VV infection and (e) 48 h after in vitro stimulation and additional shRNA-mediated knockdown of BCL2L11. (f, g) Representative dot plots and statistical analysis of the relative abundance of cotransferred WT and Batf3^{-/-} OT-I cells transduced with shRNA targeting BCL2L11 or control shRNA at d7 post transfer and VV-OVA infection. Data represents one (a n=3 at d0; n=4 at d7 and d12; n=3 at d30; b, c n=4; d n=5) or two (e n=3; g n=4) independent experiments. Error bars indicate the mean±SEM. Comparison between groups was calculated using two-tailed paired (d, g) or unpaired (e) Student's t-test *=p≤0.05; **=p<0.01; ***=p<0.001. The p-value for the RNAseq data was calculated with the “Wald test”, testing for a null hypothesis. For multiple testing, it was corrected by the Benjamini-Hochberg method, to obtain the adjusted p-value.

FIG. 5. BATF3 gain-of-function promotes CD8 T cell survival in vitro and in vivo.

(a) Polyclonal or (d) OT-I naïve CD8 T cells were isolated, activated and retrovirally transduced with pMIG-Ametrine (empty vector) or carrying the murine Batf3, pMIG-BATF3-GFP (BATF3). Cells were resting for 48 h in culture and subsequently FACS sorted and applied on the in vitro and in vivo settings. (a) Representative dotplots and statistical analysis of the in vitro kinetics of CD8 T cells coculture (50:50—empty vector: BATF3). (b) Representative histograms and statistical analysis of BCL2L11 protein levels in cocultures of CD8 T cells transduced with BATF3 (black) or empty vector (gray) after anti-CD3/CD28 stimulation. (c) Cytokine production upon PMA (5 ng/ml) plus Ionomcycin (500 ng/ml) stimulation during 4 h of CD8 T cell coculture. (d, e) Representative dotplots and statistical analysis of the in vivo kinetics of 50:50 (empty vector: BATF3) cotransferred OT-I T cells. (d) FACS analysis were performed by using blood samples from different days post VV-OVA infection and (e) splenocytes at day 30. (f) Splenocytes were stimulated with SIINFEKL (1 μg/ml) for 4 h and the cytokine production assessed by FACS. Data are representative of four (a; n=4), two (b; n=3), pool of two (c, n=9), four (d, e; n=6) and two (f; n=4) independent experiments. Error bars indicate the mean±SEM. Comparison between groups was calculated using the One sample t-test. ***=p=0.0003 (a, d); two-tailed unpaired Student's t-test (b, e)**=p<0.01; ***=p <0.001 and two-tailed paired Student's t-test (f) *=p≤0.05.

FIG. 6. BATF3 gain- of-function promotes CD8 T cell fitness.

(a) Heatmap shows the differentially expressed genes identified as p adjusted <0.01 and log 2 fold change >1.5 by comparing empty vector and BATF3 in resting and under anti-CD3/CD28 (6 h) stimulation. (b-e) Representative histograms and statistical analysis of FACS-derived data. (b) IL7R, (c) ICOS, (d) intracellular CTLA-4 and (e) Tim-3 expression in resting and after anti-CD3/CD28 stimulation of 50:50 (empty vector: BATF3) CD8 T cell coculture. (f) Aerobic glycolysis (glycoPER) and (g) oxygen consumption rate (OCR) of empty vector and BATF3 CD8 T cells single cultures in resting and 24 hours after anti-CD3/CD28 activation. Basal and maximal OCR:glycoPER ratio in (h) resting and (i) 24 h after anti-CD3/CD28 stimulation. (a) Sequencing data from 3 technical replicates. Data are representative of two independent experiments (b-i; n=3). Error bars indicate the mean±SEM. Comparison between groups was calculated using the two-tailed unpaired Student's t-test. *=p≤0.05; **=p<0.01; ***=p<0.001. The p-value for the RNAseq data was calculated with the “Wald test”, testing for a null hypothesis. For multiple testing, it was corrected by the Benjamini-Hochberg method, to obtain the adjusted p-value

FIG. 7. BATF3 is a potential target for immunotherapy in humans.

(a, b) TCR transgenic P14 naïve CD8 T cells were isolated, activated and retrovirally transduced with pMIG-Ametrine (empty vector) or carrying the murine Batf3, pMIG-BATF3-GFP (BATF3). Cells were rested for 48 h in culture, FACS sorted and contransferred into LCMV clone 13 chronically infected recipients (d30). Splenocytes were isolated at d7 post T cell transfer (d37 post infection) and analyzed by FACS. (a) Representative dotplots and statistical analysis of the ex vivo relative abundance of P14 T cells CD90.1+ (empty vector and BATF3) at day 7 post-transfer. (b) CD8 T cells were cultured for 4 h under gp33 (1 [symbol] g/ml) restimulation. Relative abundance and absolute numbers of IFNγ producing P14 CD8 T cells were analysed by FACS. (c, d) Representative dotplots and statistical analysis of (c) the numbers of cultured human CD8 T cell transduced with empty vector and murine Batf3 (empty vector and BATF3) over time and (d) intracellular cytokine staining 4 h post activation. Data are representative of two (a, b; n=14, d; n=4) or three (c; n=4) independent experiments. Error bars indicate the mean±SEM. (a, b, d) Comparison between groups was calculated using the two-tailed paired Student's t-test. ***=p<0.001 and (c) One sample t-test. ***=p=0.0002

FIG. 8

Relative abundance of leukocytes after prime (d8; VV) and recall response (d6; VV) of bone marrow chimeric mice. Data are representative of two (n=9 at prime and n=7 at recall) independent experiments. Error bars indicate the mean±SEM. Comparison between groups was calculated using two-tailed unpaired Student's t-test.

FIG. 9

Kinetic analysis of cotransferred WT (GFP) and Batf3^{-/-} (CD45.1) OT-I cells in the blood of WT recipients after L.m.-OVA i.p. Data are representative of three independent experiments (n=4). Error bars indicate the mean±SEM. Comparison between groups was calculated using the One sample t-test. ***, p=0.0003.

FIG. 10

Representative dotplots, histograms and statistical analysis of mitochondrial membrane potential of the cotransferred WT (GFP) and Batf3^{-/-} (CD45.1) OT-I cells. Cells were analysed at d8 and d12 post-VV-OVA infection. Data are representative of two independent experiments (n=4). Error bars indicate the mean±SEM. Comparison between groups was calculated using the paired Student's t-test. *=p≤0.05.

FIG. 11

(a) Kinetic analysis of cotransferred WT (CD45.1/CD45.2) and Batf3^{-/-} (CD45.1) OT-I cells in the blood of IL15 deficient (IL15^{-/-}) recipients after or VV-OVA i.p. infection. (b) qRT-PCR of Batf in ex vivo isolated WT and Batf3^{-/-} CD8 OT-I cells after VV-OVA infection, or 48 h hours post anti-CD3/CD28 in vitro stimulation. (c) Heatmap showing the 166 differentially expressed genes by comparing WT versus Batf3^{-/-} CD8 T cells in vitro stimulated with anti-CD3/CD28 for 48 h or 72 h. Columns represent technical replicates. (d) Sequence and schematic of the retroviral plasmid carrying the BcI2I11 shRNA. pLMPd-shRNA-Ametrine were provided by Transomic®. The shRNA position is marked and reported by Ametrine. The cassette carrying the shRNA is flanked by LTRs (dark grey) and MESV Psi (light grey). Ametrine is active by a PGK promoter (light grey). LTR: long terminal repeat; MESV: murine embryonic stem cell virus; Psi: RNA target site for packaging; PGK: PGK promoter. Data are representative of one experiment (n=4). Error bars indicate the mean±SEM. Comparison between groups (n=4) was calculated using the One sample t-test. **, p=0.007.

FIG. 12

For both retroviral over-expression plasmids pMIG was used as a backbone. In both cases the inserts, Batf3-IRES-GFP and Batf-IRES-GFP, were inserted between

the LTRs (dark grey) and downstream of MESV Psi (light grey) and gag (grey). As mock control pMIGR1-Ametrine was used. LTR: long terminal repeat; gag: structural precursor protein for packaging; MESV: murine embryonic stem cell virus; Psi: RNA target site for packaging.

FIG. 13

(a, b) Naïve CD8 T cells were isolated, activated and retrovirally transduced with pMIG-Ametrine (empty vector) or carrying the murine Batf3, pMIG-BATF3-GFP (BATF3) or the murine Batf, pMIG-BATF-GFP (BATF). Cells were resting for 48 h in culture after transduction and for additional 48 h after sorting before in vitro activation with anti-CD3/CD28. (a) Representative dotplots and statistical analysis of the in vitro kinetics of WT CD8 T cells coculture (50:50—empty vector: BATF3) and (b) 50:50—empty vector: BATF) in two different concentrations of IL15. (c) Representative dotplots and statistical analysis of the in vitro kinetics of WT versus Batf3^{-/-} CD8 T cells coculture (50:50—empty vector: empty vector or empty vector: BATF3). Data are representative of one (a; n=4) or two (b, c; n=4) independent experiments. Error bars indicate the mean±SEM. Comparison between groups was calculated using the One sample t-test. **, p=0.007.

FIG. 14

(a-d) Representative dotplots and gating strategy for (a) IL-7R, (b) ICOS, (c) intracellular CTLA-4 and (d) Tim-3 expression analysis in resting and after anti-CD3/CD28 stimulation of 50:50 (empty vector: BATF3) CD8 T cell coculture. (e) Statistical analysis of Glycolytic Proton Efflux Rate (GlycoPER) readouts and (f) Oxygen Consumption Rate (OCR) parameters measured in resting state and 24 h after anti-CD3/CD28 stimulation. (a-f) Data are representative of two independent experiments (n=3). Error bars indicate the mean±SEM. Comparison between groups was calculated using two-tailed unpaired Student's t-test.

### DETAILED DESCRIPTION OF INVENTION

Unless specifically defined herein, all technical and scientific terms used herein have the same meaning as commonly understood by a skilled artisan in the fields of gene therapy, immunology, biochemistry, genetics, and molecular biology.

All methods and materials similar or equivalent to those described herein can be used in the practice or testing of the present invention, with suitable methods and materials being described herein.

The term “about” used in the context of the present invention means that the value following the term “about” may vary within the range of +/−20%, preferably in the range of +1-15%, more preferably in the range of +/−10%.

All publications, patents and patent applications cited herein are hereby incorporated by reference in their entirety for all purposes. References referred to herein are indicated by a reference number in square brackets (e.g. as “[31]” or as “reference [31]”), which refers to the respective reference in the list of references at the end of the description. In case of conflict, the present specification, including definitions, will prevail over the cited references. Further, the materials, methods, and examples are illustrative only and are not intended to be limiting, unless otherwise specified.

As used herein, each occurrence of terms such as “comprising” or “comprises” may optionally be substituted with “consisting of” or “consists of”.

Lymphocyte

The present invention relates to a lymphocyte, modified to exhibit a reduced BCL2L11 level and/or increased BATF3 level. Preferably, the BCL2L11 level is reduced and/or BATF3 level increased compared to a control lymphocyte, e.g. an unmodified lymphocyte (i.e. not modified to exhibit a reduced BCL2L11 level and/or increased BATF3 level) of the same cell type from the same species, wherein the unmodified lymphocyte is preferably from the same subject as the modified lymphocyte. The comparator typically is an average BCL2L11 level and/or average BATF3 level obtained from a population of such control lymphocytes. A population of cells can be, for example, at least 10 randomly selected cells.

For example, when the modified lymphocyte is a human T cell, the BCL2L11 level can be reduced compared to an unmodified human T cell obtained from a human subject, wherein the unmodified T cell is preferably from the same human subject as the modified lymphocyte.

For example, when the modified lymphocyte is a human T cell, the BATF3 level can be increased compared to an unmodified human T cell obtained from a human subject, wherein the unmodified T cell is preferably from the same human subject as the modified lymphocyte.

BCL2L11 (“Bcl-2-like protein 11”, also known as BIM; exemplary Uniprot accession numbers 043521 (human) and 054918 (mouse)) belongs to the BCL-2 protein family and acts as pro-apoptotic regulator. The human BCL2L11 gene sequence is available under RefSeq accession number NG_029006.1. mRNA isoform 1 of human BCL2L11 is available under RefSeq accession number NM_138621.5 (SEQ ID NO: 7).

BCL2L11 is preferably mammalian BCL2L11, such as human, mouse, rat or macaque BCL2L11. More preferably, BCL2L11 is human or mouse, and most preferably human.

BATF3 (“Basic leucine zipper transcription factor ATF-like 3”; exemplary Uniprot accession numbers Q9NR55 (human; SEQ ID NO: 2) and Q9 D275 (mouse; SEQ ID NO: 3)) is a member of the AP-1 transcription factor family and binds to DNA along with c-Jun and nuclear factor of activated T cells (NFAT), thereby competing with c-Fos to form a heterodimer with c-Jun.

BATF3 is preferably mammalian BATF3, such as human, mouse, rat or macaque BATF3. More preferably, BATF3 is human or mouse, and most preferably human. Preferably, BATF3 comprises or consists of an amino acid sequence represented by SEQ ID NO: 2 or 3, or comprises or consists of an amino acid sequence that has at least 90%, at least 95% or even at least 99% sequence identity to an amino acid sequence represented by SEQ ID NO: 2 or 3 and that has an activity of reducing BCL2L11 expression.

The term “modified” as used herein in the context of a lymphocyte means that the lymphocyte is different by way of physical and/or chemical manipulation that results in a reduced BCL2L11 level and/or increased BATF3 level compared to the same lymphocyte. Thus, a “modified lymphocyte” is different from the same lymphocyte that has not been modified (by way of said physical and/or chemical manipulation that results in a reduced BCL2L11 level and/or increased BATF3 level).

Typically, the modified lymphocyte is also different from a (native) lymphocyte of the same cell type from the same species that has not been modified (to exhibit a reduced BCL2L11 level and/or increased BATF3 level). Such an unmodified lymphocyte can thus serve as comparator for the modified lymphocyte.

An unmodified lymphocyte preferably is a (native) lymphocyte of the same cell type obtainable from the same subject as the modified lymphocyte or obtainable from the same subject as the lymphocyte from which the modified lymphocyte is produced. When the modified lymphocyte is a human T cell, an unmodified lymphocyte can thus be a (native) human T cell obtainable from the same human subject as the modified T cells or obtainable from the same subject as the lymphocyte from which the modified lymphocyte is produced.

Thus, a lymphocyte that has been modified typically contains a chemical entity that is not present or that is present at different levels in said lymphocyte (or lymphocyte of the same cell type from the same species) without modification. For example, the chemical entity can be a small compound inhibitor comprised in the cell, a stretch of genomic DNA with altered nucleotide sequence compared to the unmodified cell, or a protein or RNA, such as an mRNA, miRNA, siRNA or shRNA, in the modified cell that is present at lower or higher levels in the unmodified cell.

In a preferred embodiment, the lymphocyte is modified to exhibit a reduced BCL2L11 level.

A “reduced BCL2L11 level” includes embodiments, wherein the BCL2L11 level is reduced to zero, i.e. there is no residual BCL2L11 level (e.g. by complete BCL2L11 knock-out as described herein). However, it is preferred that the BCL2L11 level is not reduced to zero (for example, by RNAi-mediated knockdown, partial knockout or an increased BATF3 level as described herein). This can be beneficial in terms of activation and functionality of the lymphocyte with a reduced BCL2L11 level. A reduced BCL2L11 level can thus mean, for example, that the BCL2L11 level is reduced to 1%-90%, preferably 1%-80%, and most preferably to 1%-50%. Thus, the BCL2L11 level can be reduced to about 20%-70%, for example. A suitable reference may be the BCL2L11 level in a control lymphocyte, e.g. an unmodified lymphocyte (i.e. not modified to exhibit a reduced BCL2L11 level and/or increased BATF3 level) of the same cell type from the same species, wherein the unmodified lymphocyte is preferably from the same subject as the modified lymphocyte. The reference typically is an average BCL2L11 level obtained from a population of such control lymphocytes.

Preferably, the reduced BCL2L11 level means that the BCL2L11 protein quantity level is reduced to 1%-90%, preferably 1%-80%, and most preferably to 1%-50%. Thus, the BCL2L11 protein quantity level can be reduced to about 20%-70%, for example. A suitable reference may be the BCL2L11 quantity level in a control lymphocyte, e.g. an unmodified lymphocyte (i.e. not modified to exhibit a reduced BCL2L11 level and/or increased BATF3 level) of the same cell type from the same species, wherein the unmodified lymphocyte is preferably from the same subject as the modified lymphocyte. The reference typically is an average BCL2L11 protein quantity level obtained from a population of such control lymphocytes.

The BCL2L11 level includes the BCL2L11 protein activity level and/or the protein quantity level, preferably the protein quantity level. Thus, the BCL2L11 level can be reduced in various ways.

For example, the BCL2L11 level can be reduced using an inhibitor of BCL2L11 protein activity.

Thus, the invention provides a lymphocyte comprising an exogenous inhibitor of BCL2L11 protein activity in the lymphocyte.

The BCL2L11 protein activity level can be determined by any known means, for example by staining for intracellular BCL2L11 protein using specific antibodies and quantification on a single cell level using flow cytometry. Together with assessing the protein quantity levels of BCL2 (anti-apoptotic factor) a ratio can be calculated, BCL2/BCL2111, that indicates BCL2L11 activity. Thus, a reduced BCL2L11 level can mean that the ratio calculated as the BCL2 protein quantity level divided by the BCL2L11 protein quantity level, BCL2/BCL2L11, in a given cell is increased (compared to a reference as defined herein). Also BCL2L11 activation leads changes in mitochondrial membrane potential and ultimately to apoptosis via activation of caspases. Both the mitochondrial membrane potential and caspase activity can be quantified using flow cytometry using standard assays/fluorescent dyes.

The term “exogenous” as used herein refers to any substance that is present in an individual living cell but that originated from outside that cell. Thus, for example, an inhibitor, activator, protein, DNA or RNA that has been produced outside a given cell and then has been introduced into the cell is an exogenous inhibitor, activator, protein, DNA or RNA.

The term “endogenous” as used herein refers to any substance that originates from within a given cell. Thus, for example, an endogenous gene, RNA or protein has not been produced outside a given cell and introduced into that cell.

The term “protein activity” as used herein means the biological activity that a given protein has in a given cell. For example, “protein activity” may refer to a (pro-apoptotic) activity of BCL2L11 in a given lymphocyte or the activity of BATF3 (e.g. of reducing BCL2L11 expression) in a given lymphocyte.

The inhibitor is preferably a specific inhibitor of BCL2L11 protein activity. Examples of inhibitors of BCL2L11 protein activity include small compound inhibitors.

The BCL2L11 level can also be reduced by increasing BCL2L11 protein degradation in the lymphocyte. This can reduce the BCL2L11 protein quantity level in the lymphocyte.

The BCL2L11 protein quantity level can be determined by any known means for protein quantification, for example, flow cytometry or quantitative western blot, preferably flow cytometry.

Thus, the invention also provides a lymphocyte comprising an exogenous activator of BCL2L11 protein degradation in the lymphocyte.

For example, protein degradation can be ubiquitin-mediated protein degradation. An activator of BCL2L11 protein degradation can be a molecule that stimulates the linkage of ubiquitin to BCL2L11.

The activator is preferably a specific activator of BCL2L11 protein degradation.

Moreover, the BCL2L11 level can also be reduced by reducing the BCL2L11 expression level in the lymphocyte. Thus, in preferred embodiments, the BCL2L11 level is a BCL2L11 expression level. Reducing the expression level in the context of the invention can reduce the protein quantity level.

The BCL2L11 expression level can be determined by any known means for protein quantification, for example, flow cytometry or quantitative western blot, preferably flow cytometry.

The term “expression level” refers to the level of gene expression from a given gene in a given cell. Gene expression in the context of protein-coding genes refers to the production of a protein via transcription of a gene encoding said protein into mRNA and translation of said mRNA into the encoded protein. Thus, the expression level can be increased by increasing transcription and/or translation from a given gene. This can be achieved e.g. by introducing activators of transcription or translation into the cell, and/or by increasing the transcription or translation of said gene in the cell by genetic modification. Conversely, the expression level can be reduced by reducing transcription and/or translation from a given gene. This can be achieved e.g. by introducing inhibitors of transcription or translation into the cell, and/or by reducing the transcription or translation of said gene in the cell by genetic modification.

The invention thus provides a lymphocyte comprising an exogenous inhibitor of BCL2L11 expression in the lymphocyte.

The inhibitor is preferably a specific inhibitor of BCL2L11 expression. An inhibitor of BCL2L11 expression can be, for example, an inhibitor of transcription from an endogenous BCL2L11 gene and/or an inhibitor of translation of an endogenous BCL2L11 mRNA.

In some embodiments, the inhibitor of BCL2L11 expression is an inhibitor of transcription from an endogenous BCL2L11 gene.

In some embodiments, the inhibitor of BCL2L11 expression is an inhibitor of translation of an endogenous BCL2L11 mRNA.

In a preferred embodiment, the invention provides a lymphocyte as described herein comprising an exogenous RNA interference (RNAi) effector molecule for reducing BCL2L11 expression in the lymphocyte.

An RNAi effector molecule refers to any molecule that is able to trigger the RNAi pathway. Such effector molecules can be, for example, siRNA, shRNAs or miRNAs. All of these molecules have in common that they contain an RNA stem formed by an antisense RNA strand and a sense RNA strand that are (partially) complementary to each other. The stem can be perfectly matched or contain mismatches. The two strands can be connected to each other by a linker that forms a loop upon hybridization of the two strands, thereby forming a stem-loop structure. An example of such a stem-loop forming molecule is an shRNA.

The antisense strand is (partially) complementary to its designated target nucleic acid sequence, preferably SEQ ID NO: 7. The RNAi effector molecule can reduce BCL2L11 expression by reducing transcription and/or translation of BCL2L11. Preferably, the RNAi effector molecule reduces translation of BCL2L11 by reducing the amount of BCL2L11 mRNA and/or by inhibiting translation (via post-transcriptional gene silencing mechanism). Typically, an RNAi effector molecule reduces the amount of target mRNA by triggering degradation by direct cleavage and/or by stimulating mRNA degradation via natural degradation pathways. It is commonly known that the so-called seed region (nucleotides 2-7 at the 5′ end of the antisense strand) is critical for gene silencing by RNAi effector molecules, and the seed region of the antisense strand should thus ideally be fully complementary to a (transcribed) portion of the target gene. Direct cleavage by RNAi factors, such as Agog, can be achieved e.g. by designing an RNAi effector comprising an antisense strand that is fully or almost fully complementary over its entire length to a (transcribed) portion of the target gene. The design and production of RNAi effector molecules against a given gene is commonly known in the art.

Thus, in an embodiment, the RNAi effector molecule is an siRNA or an shRNA for reducing BCL2L11 expression in the lymphocyte, for example, by targeting an mRNA having the sequence of SEQ ID NO: 7.

The RNAi effector molecule can be provided in the form of an exogenous RNA molecule or in the form of an exogenous DNA molecule for expressing the RNAi effector molecule. An expression cassette for an RNAi effector molecule can be a DNA molecule encoding an siRNA or an shRNA for reducing BCL2L11 expression in the lymphocyte. An expression cassette as used herein refers to a DNA sequence comprising all elements required to express a given RNA or protein to be expressed. Such elements are typically a promoter, a transcription start signal and a transcription end signal. In the case of protein expression, an expression cassette also typically contains a polyA signal and a coding sequence with a translational start site and a translational stop site.

Thus, the invention provides a lymphocyte as described herein that comprises an expression cassette for an siRNA or an shRNA for reducing BCL2L11 expression in the lymphocyte. The shRNA is preferably encoded by a nucleic acid sequence represented by any one of SEQ ID NOs: 4-6, preferably SEQ ID NO: 4, or by a nucleic acid sequence that has at least 90%, at least 95% or even at least 99% sequence identity to a nucleic acid sequence represented by any one of SEQ ID NOs: 4-6, preferably SEQ ID NO: 4, and that is able to reduce BCL2L11 expression in the lymphocyte.

The expression cassette is suitably designed to allow transcription of the RNAi effector molecule in the lymphocyte. Transcription can be, for example, constitutive or inducible. For example, an shRNA can be expressed under the control of an RNA polymerase III promoter, such as a U6 or H1 promoter. However, an shRNA can also be expressed under the control of an RNA polymerase II promoter, such as a CMV or CAG promoter. In the latter case, the shRNA is typically expressed in the form an artificial miRNA. Such an artificial miRNA typically comprises, in addition to a stem-loop as described above, 5′ and 3′ flanking sequences on the side of the stem that is opposed to the side comprising the loop. The flanking sequences can suitably allow expression as artificial primary miRNA constructs that can be cleaved by Drosha and then further be incorporated into the cellular RNAi machinery.

The invention further provides a lymphocyte as described herein, in which an endogenous BCL2L11 gene has been genetically modified in order to reduce or disrupt BCL2L11 expression. For example, the endogenous BCL2L11 gene has been genetically modified in its promoter region.

Suitable genetic modification in this context includes, for example, a complete or partial knock-out. A complete knock-out provides a complete disruption of expression from a given gene. After a partial knock-out, the expression from the gene is reduced due to the knock-out but is not completely disrupted.

A complete knock-out can be achieved by e.g. removing the entire gene (including promoter and transcribed sequence elements comprising coding and non-coding portions), removing or altering the sequence of a part of the gene that is essential for expression of the gene (e.g. (parts of the) promoter or (parts of the) coding sequence), or inserting additional elements into the gene that disrupt expression. Suitable modifications are well-known in the art.

A partial knock-out can be achieved by e.g. removing or altering the sequence of a part of the gene (e.g. (parts of the) promoter or (parts of the) coding sequence) that is essential for expression of the gene, or inserting additional elements into the gene that reduce expression. Suitable modifications are well-known in the art.

In a further embodiment, the invention provides a lymphocyte as described herein that is modified to exhibit an increased BATF3 level.

An increased BATF3 level can mean, for example, that the BATF3 level is increased at least 2-fold, at least 3-fold, at least 5-fold, or even at least 10-fold. A suitable reference may be the BATF3 level in a control lymphocyte, e.g. an unmodified lymphocyte (i.e. not modified to exhibit an increased BATF3 level) of the same cell type from the same species, wherein the unmodified lymphocyte is preferably from the same subject as the modified lymphocyte. The reference typically is an average BATF3 level obtained from a population of such control lymphocytes.

Preferably, an increased BATF3 level means that the BATF3 protein quantity level is increased at least 2-fold, at least 3-fold, at least 5-fold, or even at least 10-fold. A suitable reference may be the BATF3 protein quantity level in a control lymphocyte, e.g. an unmodified lymphocyte (i.e. not modified to exhibit an increased BATF3 level) of the same cell type from the same species, wherein the unmodified lymphocyte is preferably from the same subject as the modified lymphocyte. The reference typically is an average BATF3 protein quantity level obtained from a population of such control lymphocytes.

The BATF3 level includes the BATF3 protein activity level and/or the protein quantity level, preferably the protein quantity level. Thus, the BATF3 level can be increased in various ways.

The invention therefore provides a lymphocyte as described herein, wherein the BATF3 level is a BATF3 protein activity level in the lymphocyte.

For example, the BATF3 level can be increased using an activator of BATF3 protein activity.

Accordingly, in an embodiment, the lymphocyte comprises an activator of BATF3 protein activity in the lymphocyte. Thereby, the protein activity of BATF3 (e.g. the activity of reducing BCL2L11 expression) can be increased.

The BATF3 protein activity level can be determined by any known means, for example, by determining expression of BATF3 target genes and/or by measuring binding of BATF3 to target site in the genome of the lymphocyte. This can be done, e.g., by chromatin immunoprecipitation followed by sequencing (Chip-seq) and/or quantifying RNA levels or protein levels (for example, by western blot or flow cytometry).

The lymphocyte as described herein can also comprise an exogenous inhibitor of BATF3 protein degradation in the lymphocyte. By inhibiting BATF3 protein degradation, the protein quantity level of BATF3 can be increased.

The BATF3 protein quantity level can be determined by any known means for protein quantification, for example, flow cytometry or quantitative western blot, preferably flow cytometry.

Moreover, the BATF3 level can also preferably be a BATF3 expression level in the lymphocyte. Increasing the expression level in the context of the invention can increase the protein quantity level.

The BATF3 expression level can be determined by any known means for protein quantification, for example, flow cytometry or quantitative western blot, preferably flow cytometry.

Accordingly, the invention provides a lymphocyte as described herein comprising an exogenous activator of BATF3 expression in the lymphocyte.

The BATF3 expression level can be increased, for example, by overexpressing BATF3 in the lymphocyte.

Thus, the invention provides a lymphocyte as described herein, comprising an exogenous nucleic acid molecule for overexpressing BATF3.

Such a nucleic acid molecule is preferably an expression cassette, but can also be, for example, an mRNA encoding BATF3. Suitable promoters for use in an expression cassette for overexpressing BATF3 include a MCSV, CMV or CAG promoter, preferably MCSV. The nucleic acid molecule, when it is DNA, can be present as part of an episome or be integrated into the genome of the cell.

The lymphocyte may also comprise an exogenous activator of transcription from an endogenous BATF3 gene and/or activator of translation of an endogenous BATF3 mRNA.

Likewise, an endogenous BATF3 gene can be genetically modified in order to increase BATF3 expression in the lymphocyte. Preferably, the endogenous BATF3 gene has been genetically modified in its promoter region. For example, the endogenous promoter can be replaced by a stronger promoter that provides increased transcription in the lymphocyte so modified.

In view of the above, the invention provides a lymphocyte as described herein,
a) comprising an exogenous inhibitor of BCL2L11 protein activity in the lymphocyte; and/or
b) comprising an exogenous activator of BCL2L11 protein degradation; and/or
c) comprising an exogenous inhibitor of BCL2L11 expression in the lymphocyte and/or an exogenous expression cassette for expressing an inhibitor of BCL2L11 expression in the lymphocyte;
and/or
d) in which an endogenous BCL2L11 gene has been genetically modified in order to reduce or disrupt BCL2L11 expression, wherein optionally the endogenous BCL2L11 gene has been genetically modified in its promoter region.

Moreover, the invention provides a lymphocyte as described herein,
a) comprising an exogenous activator of BATF3 protein activity in the lymphocyte; and/or
b) comprising an exogenous inhibitor of BATF3 protein degradation; and/or
c) comprising an exogenous activator of BATF3 expression in the lymphocyte and/or a exogenous nucleic acid molecule for overexpressing BATF3 in the lymphocyte; and/or
d) in which an endogenous BATF3 gene has been genetically modified in order to increase BATF3 expression, wherein optionally the endogenous BATF3 gene has been genetically modified in its promoter region.

The lymphocyte of the present invention is preferably a mammalian lymphocyte, such as human, mouse, rat or macaque. More preferably, the lymphocyte is a human or mouse lymphocyte, and most preferably a human lymphocyte.

Lymphocytes include, for example, T cells, B cells and innate lymphoid cells, such as natural killer (NK) cells, ILC1, ILC2 and ILC3 cells. The lymphocyte is preferably a T cell or an innate lymphoid cell, such as and ILC1, NK cell, ILC2 or ILC3 (see, e.g. Cell 2018; 174(5):1054-1066 describing such cells; incorporated herein by reference). More preferably, the lymphocyte is a T cell or NK cell, and most preferably a T cell.

A T cell can be, for example, a CD8+ T cell or a CD4+ T cell, preferably a CD8+ T cell.

The lymphocyte preferably is a lymphocyte that expresses an endogenous (physiologic) T cell receptor (TCR).

Even more preferably, the lymphocyte has been modified to express a transgenic TCR. Even more preferably, the lymphocyte is a cell expressing a chimeric antigen receptor (CAR). A CAR is a (not naturally occurring) receptor that can be expressed on the surface of a cell and that can bind to a ligand, e.g. expressed on the surface of another cell. The receptor can thereby lead to recruitment of a cell expressing the receptor to target cells that express the ligand on their surface. Moreover, upon binding to the ligand the CAR can optionally transmit an intracellular signal within the cells on which it is expressed. Thus, for example, the CAR can be expressed on a T cell and activate the T cell upon binding to its ligand. The lymphocyte is preferably a T cell expressing a CAR, such as a CD8+ T cell or a CD4+ T cell expressing a CAR.

It has been found by the present inventors that reduced levels of BCL2L11 and/or increased levels of BATF3 can lead to increased survival of lymphocytes in vivo. Without wishing to be bound by any theory, it is hypothesized that the increased survival is due to reduction of pro-apoptotic signals by BCL2L11. Moreover, the present inventors also found that a reduced BCL2L11 level in the sense that the lymphocyte still has a residual level of BCL2L11 (e.g. by RNAi-mediated knockdown or by an increased BATF3 level) is advantageous in a therapeutic setting over eliminating BCL2L11 levels entirely (e.g. by a full genetic knockout). For example, a reduced BCL2L11 level in the sense that the lymphocyte still has a residual level of BCL2L11 can be advantageous regarding lymphocyte activation and functionality. Interestingly, the inventors did not observe increased survival outside a living organism. Thus, the increased survival could not have been predicted based on cell culture experiments. Increased levels of BATF3 confer increased survival of lymphocytes also in culture systems, demonstrating that BATF3 promotes lymphocyte survival beyond its function to modulate BCL2L11.

Accordingly, the invention also provides a lymphocyte as described herein, wherein the lymphocyte exhibits increased survival in vivo. Survival in vivo can be determined, for example, by determining the number of lymphocytes of the invention per ml in a (peripheral) blood sample obtained from a subject at a predetermined time point after administration, e.g. 8 days after administration. (Peripheral) Blood samples can be taken at two or more different time points to monitor the number of lymphocytes of the invention per ml in the blood samples over time.

The invention also provides a lymphocyte as described herein, wherein the lymphocyte exhibits increased survival in a subject, preferably a human subject. For example, increased survival can be observed after administration to the subject.

Increased survival is preferably seen in comparison to a control lymphocyte, e.g. an unmodified lymphocyte (i.e. not modified to exhibit a reduced BCL2L11 level and/or increased BATF3 level) of the same cell type from the same species, wherein the unmodified lymphocyte is preferably from the same subject as the modified lymphocyte. The comparator typically is an average survival of a population of such control lymphocytes.

Thus, increased survival can be determined, for example, by comparing the survival in vivo of the lymphocytes of the invention with the survival in vivo of control lymphocytes in the same subject. For example, the number of lymphocytes of the invention per ml and the number of control lymphocytes per mL in the same (peripheral) blood sample obtained from a subject can be compared at a predetermined time point after administration, e.g. on day 0, day 1, day 3, day 7, day 14, day 21 and/or day 28 after administration. For example, the number of lymphocytes of the invention can be at least 2 times higher, such as at least 3, 4 or 5 times higher, preferably at least 10 times higher than the number of control lymphocytes in the same blood sample taken on day 7, day 14, day 21 and/or day 28 after administration, preferably on day 7 after administration. The number of lymphocytes of the invention can be at least 2 times higher, such as at least 3, 4 or 5 times higher, preferably at least 10 times higher than the number of control lymphocytes in the same blood sample taken at even later time points, such as 2, 3, 4, 5, 6 or even 12 months after administration.

Moreover, IL7R (also known as CD127) can be used as a marker for lymphocyte survival in vivo. Thus, the lymphocyte of the invention can also exhibit increased expression of IL7R in a subject (compared to an unmodified lymphocyte of the same cell type from the same species, optionally from the same subject), preferably a human subject. For example, increased expression of IL7R can be observed after administration to the subject. Expression of IL7R can be determined by any known means, such as flow cytometry, quantitative Western Blot, or-on the RNA level-by quantitative PCR, such as quantitative reverse transcription PCR (qRT-PCR).

A “subject” in the context of the invention is preferably mammalian, such as human, mouse, rat or macaque. Preferably, it is a human subject.

The subject is preferably a (human) subject diagnosed with a disease to be treated using the modified lymphocyte.

Method for Producing Lymphocytes

The present invention also relates to a method for producing lymphocytes as defined herein. The disclosure of the lymphocyte as such can be applied to the lymphocyte to be produced by the method mutatis mutandis.

For example, the invention provides a method for producing a lymphocyte that exhibits a reduced BCL2L11 level and/or increased BATF3 level, wherein the method comprises
a) introducing into the lymphocyte an exogenous inhibitor of BCL2L11 protein activity in the lymphocyte; and/or
b) introducing into the lymphocyte an exogenous activator of BCL2L11 protein degradation in the lymphocyte; and/or
c) introducing into the lymphocyte an exogenous inhibitor of BCL2L11 expression in the lymphocyte and/or an exogenous expression cassette for expressing an inhibitor of BCL2L11 expression in the lymphocyte; and/or
d) genetically modifying an endogenous BCL2L11 gene in order to reduce or disrupt BCL2L11 expression, wherein optionally the endogenous BCL2L11 gene has been genetically modified in its promoter region; and/or
e) introducing into the lymphocyte a exogenous activator of BATF3 protein activity in the lymphocyte; and/or
f) introducing into the lymphocyte an exogenous inhibitor of BATF3 protein degradation; and/or
g) introducing into the lymphocyte an exogenous activator of BATF3 expression and/or an exogenous nucleic acid molecule for overexpressing BATF3 in the lymphocyte; and/or
h) genetically modifying an endogenous BATF3 gene in the lymphocyte in order to increase BATF3 expression, wherein optionally the endogenous BATF3 gene has been genetically modified in its promoter region.

For example, the invention provides a method for producing a lymphocyte that exhibits a reduced BCL2L11 level, wherein the method comprises
a) introducing into the lymphocyte an exogenous inhibitor of BCL2L11 protein activity in the lymphocyte; or
b) introducing into the lymphocyte an exogenous activator of BCL2L11 protein degradation in the lymphocyte; or
c) introducing into the lymphocyte an exogenous inhibitor of BCL2L11 expression in the lymphocyte and/or an exogenous expression cassette for expressing an inhibitor of BCL2L11 expression in the lymphocyte; or
d) genetically modifying an endogenous BCL2L11 gene in order to reduce or disrupt BCL2L11 expression, wherein optionally the endogenous BCL2L11 gene has been genetically modified in its promoter region.

For example, the invention provides a method for producing a lymphocyte that exhibits an increased BATF3 level, wherein the method comprises
a) introducing into the lymphocyte a exogenous activator of BATF3 protein activity in the lymphocyte; or
b) introducing into the lymphocyte an exogenous inhibitor of BATF3 protein degradation; or
c) introducing into the lymphocyte an exogenous activator of BATF3 expression and/or an exogenous nucleic acid molecule for overexpressing BATF3 in the lymphocyte; or
d) genetically modifying an endogenous BATF3 gene in the lymphocyte in order to increase BATF3 expression, wherein optionally the endogenous BATF3 gene has been genetically modified in its promoter region.

In an embodiment of the present invention, the method for producing a lymphocyte comprises a step of isolating immune cells from a blood sample of a subject, before modifying the lymphocyte.

The blood sample is preferably derived from a human subject, preferably a human subject diagnosed with a disease to be treated using the modified lymphocyte, such as cancer, an infectious disease or an autoimmune disease, such as a chronic inflammatory disease or degenerative disease.

The method may further comprise formulating the lymphocytes into a formulation that is suitable for administration to a human subject.

In other embodiments, the lymphocyte is modified in vivo. Thus, the invention also provides a method for producing immune cells, comprising administering a vector suitable for modifying a lymphocyte (such as a T cell) according to the present invention to a subject (in vivo gene transfer), see e.g. references [51]-[53], all incorporated by reference. Preferably, the expression vector for in vivo gene transfer is a lentiviral vector pseudotyped to transduce human lymphocytes (preferably T cells), or a nanoparticle containing a non-viral vector suitable for delivering the non-viral vector to human lymphocytes (preferably T cells).

The nucleic acid molecules used in the present invention, such as expression cassettes, can be in the form of expression vectors.

A wide range of expression vectors for polypeptides as well as non-coding RNAs, such as siRNA or shRNAs, are known in the art and are further detailed herein. For example, in some embodiments of the invention, the expression vector is a non-viral or viral vector, and-in the context of medical purposes—preferably a non-viral vector.

The expression vector can be a minimal DNA expression cassette. Moreover, an expression vector may be a DNA expression vector such as a plasmid, linear expression vector or an episome. In certain aspects, the vector comprises additional sequences, such as sequences that facilitate expression of the polypeptide, such as a promoter, enhancer, poly-A signal, and/or one or more introns. In certain aspects, the expression vector may be a transposon donor DNA molecule, preferably a minicircle DNA.

The present invention also relates to minicircle DNA comprising a polynucleotide of the present invention as defined herein. As used herein, the term “minicircle DNA” refers to vectors which are supercoiled DNA molecules that lack a bacterial origin of replication and an antibiotic resistance gene. Therefore, they are primarily composed of a eukaryotic expression cassette.

In a useful embodiment the minicircle DNA of the invention is introduced into the cell in combination with mRNA encoding a transposase protein by electrotransfer, such as electroporation, nucleofection; chemotransfer with substances such as lipofectamin, fugene, calcium phosphate; nanoparticles, or any other conceivable method suitable to transfer material into a cell.

A viral vector can be, for example, a gamma retroviral vector or a lentiviral vector. Such vectors and their construction and production are commonly known in the art.

The polynucleotide or expression vector can be introduced into lymphocytes by any suitable means, such as by transfection or by transduction. Transfection refers to chemical or physical delivery into the cells, e.g. by electrotransfer, such as electroporation, nucleofection; chemotransfer with substances such as lipofectamin, fugene, calcium phosphate, PEI. Transduction refers to other means of (targeted) delivery into the cells including delivery by a viral vector or nanoparticles. However, the present invention is not limited to any particular method of delivery of genetic material into immune cells, such that also any other conceivable method suitable to transfer genetic material into a cell can be used in the context of the invention.

Typically, the polynucleotide or expression vector used in the context of the invention allows stable expression of the encoded transgene (such as an anti-BCL2L11 shRNA or a BATF3 coding sequence). Stable expression in this context means that the transgene and expression thereof is not lost when the cells comprising the same proliferate. Stable expression can be achieved, e.g., by expression cassettes that are integrated into the genome of the host cell, such as a lymphocyte of the invention. Moreover, suitable promoters are known in the art that allow prolonged expression of a transgene also in vivo.

The invention also provides a lymphocyte or formulation obtainable by the method of producing a lymphocyte as described herein.

Pharmaceutical Composition

The present invention also relates to a composition comprising a plurality of lymphocytes as described herein. The composition is preferably a pharmaceutical composition. The composition may further comprise at least one pharmaceutically acceptable carrier.

In one embodiment of the invention, the pharmaceutical composition may be formulated as infusion solution comprising NaCl, glucose and human serum albumin in an amount of 0.45%, 2, 5% and 1%, respectively.

Medical Uses

The present invention also relates to the lymphocyte or composition as described herein for use as a medicament.

The type of disease that can be treated by a lymphocyte of the invention is not particularly limited. This is because the present invention provide a general means of extending the in vivo activity of the modified lymphocytes. Thus, the invention can be applied to any type of disease that is amenable to immunotherapies using lymphocytes. Such diseases include, for example, cancer, infectious diseases and autoimmune diseases.

Accordingly, the invention provides a lymphocyte or composition as described herein for use in a method of treating cancer, an infectious disease or an autoimmune disease, such as a chronic inflammatory disease or degenerative disease, preferably cancer.

The invention also provides a method of treating cancer, an infectious disease or an autoimmune disease, such as a chronic inflammatory disease or degenerative disease, using a lymphocyte or composition as described herein, preferably cancer.

Cancer includes all known malignancies, e.g. hematologic malignancies such as leukemia, lymphoma, multiple myeloma; solid tumors such as breast cancer, lung cancer, pancreatic cancer.

Infectious diseases include but are not limited to viral infections, e.g. Hepatitis B, HIV; fungal infections, e.g. aspergillosis; bacterial infections; infections with other pathogens.

Autoimmune disease includes, e.g. chronic inflammatory diseases or degenerative diseases.

Chronic inflammatory diseases include e.g. Crohn's disease, Encephalitis disseminata.

Degenerative diseases include e.g. Alzheimer's disease.

For example, the (use in) the method of treatment can comprise the administration of the lymphocyte or composition to a subject (in need thereof), preferably a human subject.

The lymphocyte of the invention may exhibit an improved therapeutic activity and/or provide an improved therapeutic outcome.

For example, the improved therapeutic activity may be evidenced by faster and/or more pronounced reduction of tumor size or viral (e.g. LCMV) load.

Moreover, the modifications as described herein may lead to improved engraftment and persistence of the lymphocyte or improved pharmacokinetics thereof, e.g. evidenced by
greater AUC;
longer half-life (of the lymphocyte in vivo);
superior total time period of survival in vivo;
shorter time period from adoptive transfer to peak level (of modified lymphocyte numbers);
longer time period at or near peak level (of modified lymphocyte numbers)
longer time period of modified lymphocyte numbers above the mean number of modified lymphocytes during the course of treatment;
longer time period from peak level (of modified lymphocyte numbers) to disappearance (of modified lymphocytes).

Area-under-curve (AUC) in this context relates to integral of a curve that describes the absolute or relative number of modified lymphocytes in peripheral blood (or another anatomical compartment, e.g. bone marrow, cerebrospinal fluid) over time.

The number of (modified) lymphocytes are typically determined by determining the number of (modified) lymphocytes per ml in a (peripheral) blood sample obtained from a subject at a given time point after treatment (e.g. administration of the modified lymphocytes). The peak level and disappearance of (modified) lymphocytes can be determined by a time course, i.e. determining the number of (modified) lymphocytes at different time points after treatment. The peak level corresponds to the maximal number of (modified) lymphocytes during the course of the treatment, e.g. as determined by a time course using peripheral blood samples, in which the number of modified lymphocytes is optionally determined once per day. Likewise, the mean number of (modified) lymphocyte during the course of the treatment may be calculated as the mean value of the numbers determined by such a time course.

In an embodiment of the invention, the lymphocyte or composition is to be administered intravenously.

The pharmaceutical composition as described above comprising the modified lymphocytes are stored at 2-8° C. The pharmaceutical composition is stable for (at least) 48 hours after formulation and ought to be administered to the patient within this period.

### EXAMPLES

CAR-T cells as generated in the experimental section of the application relate to a non-limiting exemplified embodiment of the present invention.

Materials and Methods

Animals

Wildtype C57BL/6 J and TCR transgenic OT-I and Batf3^{-/-} mice [15] were originally purchased from Jackson or Janvier Labs. P14 and XCR1-DTR-Venus mice [32] were provided by H. C. Probst and T. Kaisho, respectively. All mice were maintained in specific pathogen-free conditions at an Association for Assessment and Accreditation of Laboratory Animal Care-accredited animal facility. All procedures were approved by the North Rhine-Westphalia State Environment Agency (LUA NRW) and/or the Lower Franconia Government.

Generation of Mixed Bone Marrow Chimeric Mice

The isolated bone marrow cells were mixed in a 1:1 ratio and a total of 2×10⁶ cells of each type (WT and Batf3^{-/-}) and transferred i.v. into the 9 Gy irradiated wild type mice. Chimerism was confirmed eight weeks after transplantation.

Treatment of Mice

For LCMV clone 13 infection, C57BL/6J mice were intraperitonally injected with 300 μg of anti-CD4 antibody clone GK1.5 (BioXCell) twice before infection (day-2 and day 0). For depletion of XCR1⁺ DC, transgenic mice and control littermates were treated with 0.5 μg DTX i.p. (Merck Millipore) on d-1, d0 and d1.

Infections

10⁶ PFU VV-OVA, 10⁵ PFU Influenza or 10⁵ LM-OVA were diluted in PBS and injected intraperitoneally, intranasally or intravenously, respectively. 2×10⁶ LCMV clone 13 were diluted in PBS and injected intravenously.

Adoptive Naïve T Cell Transfer

OT-I, and OT-I Batf3^{-/-} CD8⁺ T cells were sorted using a MACS CD8⁺ T cell negative selection kit (Miltenyi) combined with biotinylated anti-CD44 (IM7, BD Biosciences). 5×10³ cells were transferred i.v.

Retroviral Vectors and shRNAs

pMIG-Batf3-IRES-GFP was applied for overexpression of BATF3. Batf3 was sub-cloned from a synthetic gene fragment plasmid (courtesy to Invitrogen GeneArt) into pRP backbone. From the resulting pRP-Batf3 plasmid, Batf3 was cut using single cutting restriction enzymes Bglll and Xhol and ligated into pMIG, generating pMIG-Batf3-IRES-GFP. pMIG was a gift from William Hahn (Addgene plasmid #9044; http://n2 t.net/addgene:9904; RRID: Addgene_9904). pMIG-Batf-IRES-GFP was applied for overexpression of BATF. Batf was subcloned from pCMV6-Entry-Batf (MR222114, OriGene®) by PCR and ligated into pMIG opened with restriction enzymes Xhol and EcoRl. MigR1-mAmetrine was used as an empty-vector control. For the knockdown of BCL2L11, shRNAs were used. The shRNAs for BcI2I11 as well as a non-targeted control shRNA were expressed by the pLMPd-Ametrine backbone (TRMSU2002, TransOmics®). The three shRNAs were encoded by the following DNA sequence, wherein shRNA (3) is preferred:

shRNA (1):
(SEQ ID NO: 5)
5′TGCTGTTGACAGTGAGCGCAGCAACCTTCTGATGTAAGTTTAGTGAA
GCCACAGATGTAAACTTACATCAGAAGGTTGCTTTGCCTACTGCCTCGG
A-3′
shRNA (2):
(SEQ ID NO: 6)
5′TGCTGTTGACAGTGAGCGAAGTTGTGACAAGTCAACACAATAGTGAA
GCCACAGATGTATTGTGTTGACTTGTCACAACTCTGCCTACTGCCTCGG
A-3′
shRNA (3):
(SEQ ID NO: 4)
5′TGCTGTTGACAGTGAGCGAAAGCAACCTTCTGATGTAAGATAGTGAA
GCCACAGATGTATCTTACATCAGAAGGTTGCTTGTGCCTACTGCCTCGG
A-3′

Retroviral Transduction, Adoptive Transfer of Transduced OT-I and P14 T Cells and In Vitro Cocultures of Murine CD8 T Cells

Total CD8⁺ T cells were isolated using a mouse CD8 T Cell negative Enrichment kit (STEMCELL Technologies) and stimulated with 1 μg/ml plate-bound anti-CD3e (clone 2C11) plus 1 μg/ml anti-CD28 Abs (clone 37.51, both BioXCell) at a density of 2×10⁶ cell/ml in 12 well plates. T cells were transduced 24 hrs after stimulation by spin-infection (2.500 rpm, 32° C., 90 min) in the presence of frozen retroviral supernatant with 10 μg/ml polybrene (SantaCruz Biotechnologies). Retroviral supernatant was produced in the Platinum-E retroviral packaging cell line. Platinum-E cells were transfected by lipofection using GeneJet (Signagen) with various retroviral expression plasmids and the amphotrophic packaging vector pCL-10A1 (Addgene). The supernatant was collected two and three days after transfection, filtered and stored in aliquots at −80° C. After spin-infection, viral supernatant was removed from the CD8⁺ T cells and replaced by fresh media. 24 hrs after transduction, T cells were transferred into 6 well plates with fresh media containing 50 ng/ml IL-15 and maintained for two days at a density of 0.5×10⁶ cells/ml. Transduced cells were FACS-sorted using a sterile FACSAria III cell sorter (BD Biosciences) and cultivated for additional 48 hrs in fresh media containing 50 ng/ml IL-15 at a density of 0.5×10⁶ cells/ml. At day 6 of culture, pMIG-Ametrine and pMIG-BATF3-GFP transduced memory CD8⁺ T cells were mixed at a 1:1 ratio and re-stimulated with 1 μg/ml plate-bound anti-CD3e (clone 2C11) plus 1 μg/ml anti-CD28 Abs (clone 37.51, both BioXCell) for the in vitro experiments, or transferred (P14 tdTomato cells) intravenously into LCMV-infected C57BL/6J host mice. Transduced OT-I CD45.1 were transferred into naïve C57BL/6J mice followed by VV-OVA i.p. infection. A total of 1,5×10⁶ transduced CD8 T cells (50/50—empty vector/BATF3) were transferred into the hosts in both, P14 and OT-I adoptive transfer experimental setup.

Isolation and Cultivation of Primary Human Samples.

Blood samples were obtained from healthy donors after provided written informed consent to participate in research protocols approved by the Institutional Review Board of the University Hospital of Würzburg. Peripheral blood mononuclear cells (PBMC) were isolated by Biocoll centrifugation (Biochrom, Merck Millipore, Darmstadt, Germany). Bulk CD8⁺ T cells were isolated by untouched magnetic cell separation (Miltenyi Biotec, Bergisch Gladbach, Germany). Primary human T cells were cultured in RPMI-1640 (supplemented with 1% GlutaMAX®, 50 mM 2-mercaptoethanol, 100 U/mL penicillin/streptomycin (all Gibco, ThermoFisher, Waltham, MA, USA) and 10% human serum (Deutsches Rotes Kreuz) containing 50 U/mL hIL-2 (Miltenyi Biotec, Bergisch Gladbach, Germany).

Retroviral Transduction of Human CD8 T Cells

Retroviral supernatant was produced as described previously, using the Platinum-A packaging cell line to obtain amphotropic retrovirus (see retroviral transduction session). CD8⁺ T cells were isolated and stimulated for 18 hours using human T-Activator CD3/CD28 Dynabeads (ThermoFisher, Waltham, MA, USA). On the next day, CD8⁺ T cells were collected, resuspended in retroviral supernatant containing 10 μg/mL polybrene (Merck Millipore, Darmstadt, Germany) and transduced by spin-infection (800×g, 32° C., 45 min). 24 hours after transduction, a half-medium change was performed. CD8⁺ T cells were maintained in fresh medium at a density of 2×10⁶ cells/mL. CD3/CD28 beads were removed 96 hours after transduction and transduction efficiencies were determined by flow cytometry using a FACS Canto II (BD Bioscience). On day 7, CD8⁺ T cells were sorted for their respective reporter gene expression using a FACSAria III cell sorter (BD Bioscience) and cultivated for another several days in fresh medium containing 50 U/mL hIL-2 plus at a density of 0.5×10⁶ cells/ml. Cells were cultured in 1:1 ratio (empty vector:mBATF3) and resting or with CD3/CD28 Dynabeads.

Murine T Cell Cultures

CD8⁺ T cells were isolated from single cell suspensions of spleens and lymph nodes by negative separation using a mouse CD8⁺ T cell Enrichment kit (STEMCELL Technologies). 2×10⁶ T cells were stimulated in 2 ml RPMI 1640 medium (supplemented with 10% FBS, 2 mM L-glutamine, 50 mM 2-mercaptoethanol and 100 Wml penicillin plus streptomycin; all Gibco) with 1 μg/ml plate-bound anti-CD3 (clone 2C11) plus 1 μg/ml anti-CD28 Abs (clone 37.51, both BioXCell) in 12 well plates. 48 hrs after activation, T cells were transferred into 96 well plates with fresh media containing 50 ng/ml IL-15 (PeproTech) and maintained for several days at a constant density of 0.5×10⁶ cells/ml with fresh medium and cytokines.

Extracellular Flux Analysis (Seahorse Assay)

Oxygen consumption rates (OCR) and glycolytic proton efflux rates (glycoPER) were measured using an oxygen controlled XFe96 Extracellular Flux Analyzer (Seahorse Bioscience). 1.5×10⁵CD8⁺ T cells per well were seeded on Cell-Tak (Corning) in 6-8 replicates in XF media (Seahorse Biosciences) supplemented with 10 mM glucose (Sigma Aldrich), 2 mM GlutaMAX (Gibco) and 1 mM sodium pyruvate (Corning). The cells were incubated for one hour in a non-CO2 incubator at 37° C. before oxygen consumption and extracellular acidification were analyzed under basal conditions and after the following treatments: ATP synthase inhibitor oligomycin (2 μM), 2-desoxy-glucose (50 mM) to inhibit glycolysis, the protonophore Carbonyl cyanide-4-(trifluoromethoxy)phenylhydrazone (FCCP) (1 μM) to uncouple mitochondria, the mitochondrial complex I inhibitor rotenone (500 nM) and the mitochondrial complex III inhibitor antimycin A (500 nM). Basal OCR was calculated by subtracting the OCR after rotenone and antimycin A treatment from the OCR before oligomycin treatment. Maximal OCR was calculated by subtracting the OCR after rotenone and antimycin A treatment from the OCR measured after addition of FCCP. Basal glycoPER was calculated by subtracting the values after 2-DG from the glycoPER values before oligomycin treatment. Maximal glycoPER was calculated by subtracting values after 2-DG from the glycoPER values following oligomycin treatment. Ratios of OCE to glycoPER were calculated under basal and maximal conditions to determine the metabolic phenotypes of CD8⁺ T cells.

Mitochondrial Potential Measurement by Flow Cytometry

Cells were stained with MitoTracker® Deep Red FM (250 nM) or TMRE (70 nM) (Thermofisher) in an incubator at 37 C (5% CO2) for 30 min prior cell surface staining. Cells were then washed three times with FACS buffer (PBS 2% FCS) and followed by cell surface staining for further FACS analysis. FCCP (1μM) treatment was used to disrupt the mitochondrial membrane potential and then serving as negative control.

Flow Cytometry (Surface and Intracellular Staining)

Single cell suspensions obtained from spleens or blood were used for flow cytometry analysis. Cells were surface stained with anti-CD8 (Biolegend, clone 53-6.7), anti-CD44 (Biolegend, clone IM7), anti-CD127 (eBioscience, clone A7R34), anti-KLRG1 (eBioscience, clone 2F1), anti-CX3CR1 (Biolegend, clone SA011F11), anti-ICOS (BD Biosciences, clone C398.4A), anti-Tim-3 (Biolegend, clone RMT3-23). For intracellular staining cells were first fixed with IC Fixation Buffer (eBioscience 00-8222-49) for 30 min on ice. Antibodies were diluted in Permeabilization Buffer 1× (eBioscience 00-8333-56). anti-IFNγ (Biolegend, clone XMG1.2), anti-TNFα (Biolegend, clone MD6-XT22), anti-IL2 (Biolegend, clone JES6-5H4), anti-CTLA4 (Biolegend, clone UC10-4B9), anti-BCL2L11 (Cell Signalling, clone C34C5) plus anti-rabbit (Life Technologies, clone A-31573). For anti-Ki67 staining (BD Biosciences, clone B56), cells were fixed with eBioscience™ Foxp3/Transcription Factor Staining Buffer (00-5523-00).

RT-PCR

RNA was isolated from CD8 T cells (see Murine T cells culture session) using the RNeasy Micro Kit (Qiagen) following the manufacturer's instructions. RNA was converted to cDNA using the iScript™ cDNA Synthesis Kit (Bio-Rad 1708891) according to manufacturer's instructions. Real-time PCR was performed on the Bio-Rad CFX connect Real-time PCR instrument (Bio-rad) using the BATF3 TaqMan® Gene Expression Assay (Catalog 4331182).

RNA-Sequencing

At day 6 of culture, pMIG-Ametrine and pMIG-BATF3-GFP transduced memory CD8⁺ T cells were maintained at a 0.5×10⁶ cells/ml density (see the Retroviral Transduction and Murine cell culture sessions). Three technical replicates were kept resting or re-stimulated with 1 μg/ml plate-bound anti-CD3e (clone 2C11) plus 1 μg/ml anti-CD28 Abs (clone 37.51, both BioXCell) in vitro for 6 hours. Cells were harvested, and total mRNA was isolated with the RNeasy Mini Kit (Qiagen) following the manufacturer's instructions. RNA quality was checked using a 2100 Bioanalyzer with the RNA 6000 Pico kit (Agilent Technologies). The RIN for all samples was ˜7.6. DNA libraries suitable for sequencing were prepared from 100 ng of total RNA with oligo-dT capture beads for poly-A-mRNA enrichment using the TruSeq Stranded mRNA Library Preparation Kit (Illumina) according to manufacturer's instructions. After 15 cycles of PCR amplification, the size distribution of the barcoded DNA libraries was estimated ˜320 bp by electrophoresis on Agilent High Sensitivity Bioanalyzer microfluidic chips. Sequencing of pooled libraries, spiked with 1% PhiX control library, was performed in single-end mode on the NextSeq 500 platform (Illumina) with the High Output Kit v2.5 (75 Cycles). Demultiplexed FASTQ files were generated with bcl2fastq2 v2.20.0.422 (Illumina).

RNA-Seq Data Processing and Analysis

The obtained FASTQ files were aligned using STAR 2.7 to the GRCm38.98 reference genome using standard settings [48]. The aligned data were counted using the featureCounts function of the Rsubread package in R [49]. Differential expression analysis based on the raw count-matrix was performed using DESeq2 [50]. Significant differentially expressed genes were defined as having an adjusted p-value <0.01 and log 2FoldChange >1.5. All significant differentially expressed genes, were divided into six categories, based on their expression behavior across all samples and visualized using the pHeatmap package in R https://CRAN.R-project.org/package=pheatmap.

Generation of CD8⁺ Tissue Resident Memory T Cells (T_{RM})

Naïve (WT tdTomato and Batf3^{-/-} GFP) T cell receptor transgenic CD8⁺ T cells (OT-I) were transferred into wildtype recipient mice followed by 1×10⁶ PFU VV-OVA i.p. infection (prime). At the peak of T cell response (day 8), both ears were intradermally infected with 1 ×10⁷ IU MVA-OVA (pull). T_{RMs} were analysed by FACS and under 2-photon microscope at day 40 post-MVA-OVA infection. Lymhocytes isolation from skin was performed by applying an enzymatic digestion-based protocol.

Statistical Analysis

Apart of the genomic data, all for the biological data were analysed using Prism 8 software (GraphPad) by two-tailed paired Student's t-test, two-tailed unpaired Student's t-test, or one- and two-way ANOVA test.

Results

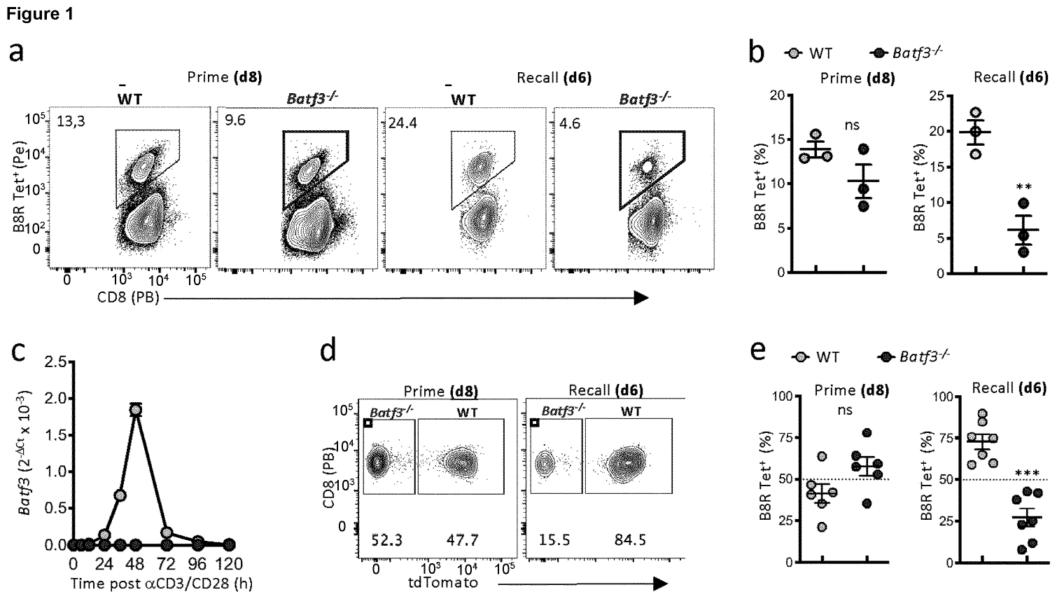

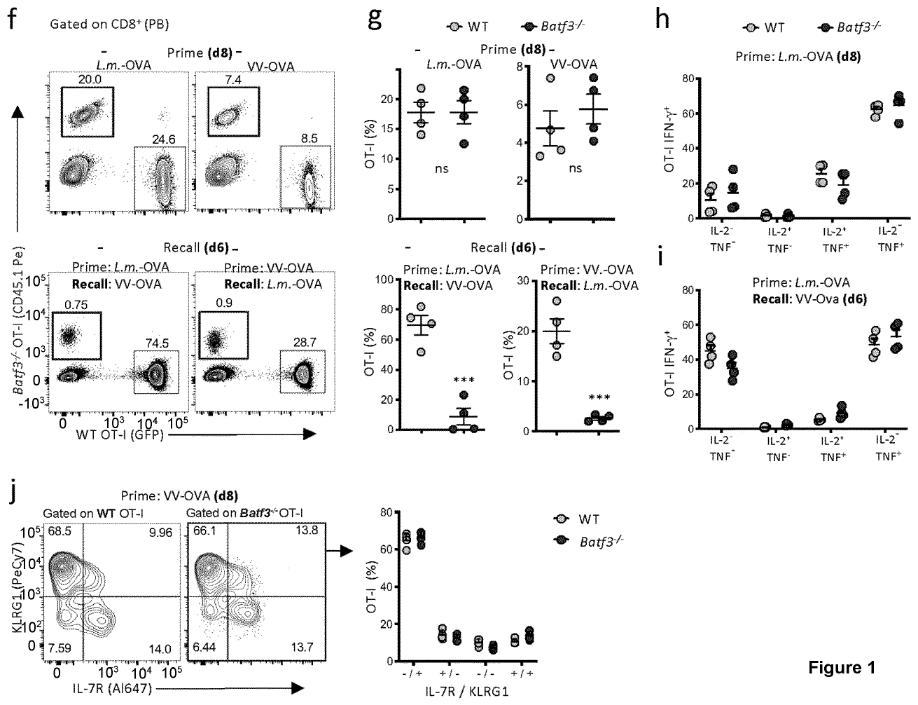

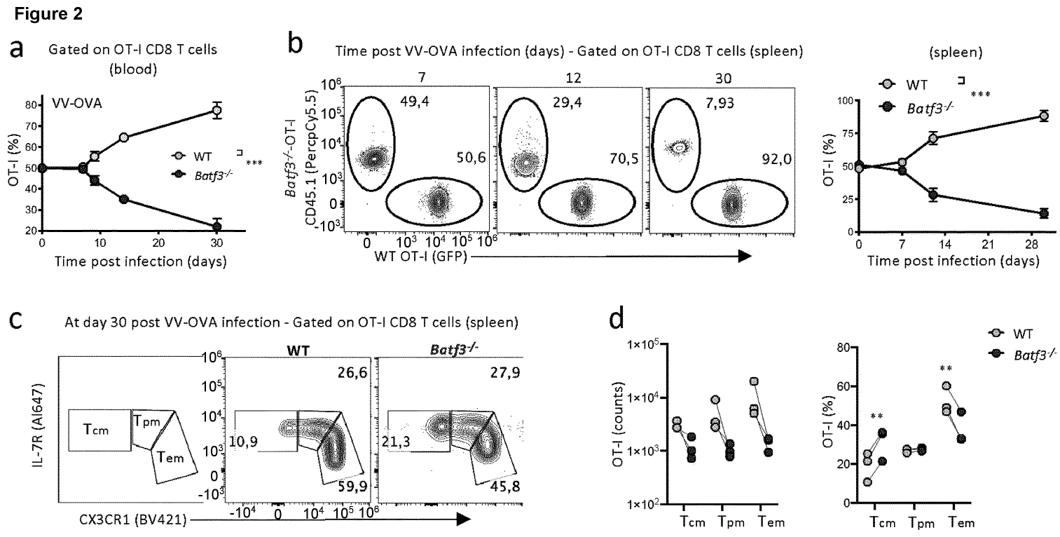

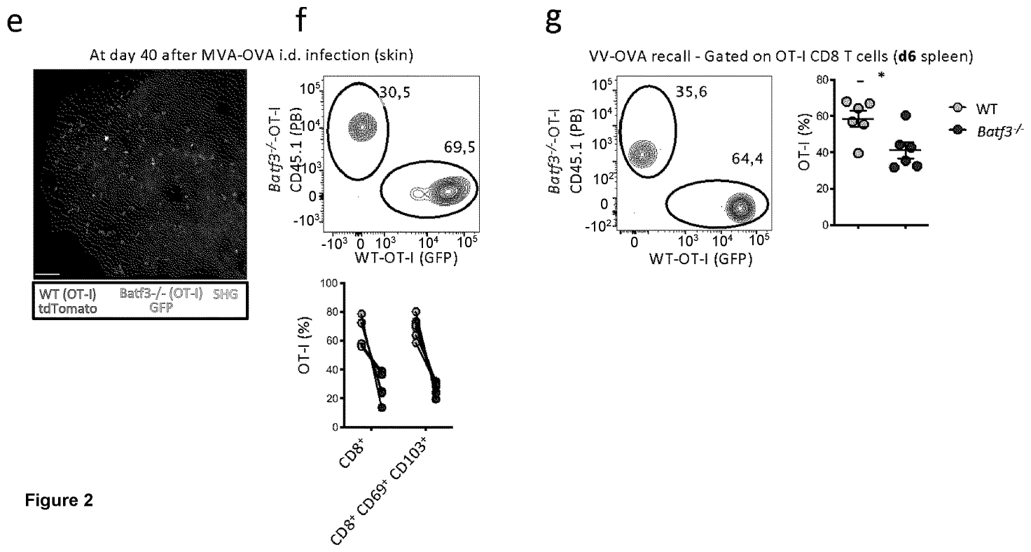

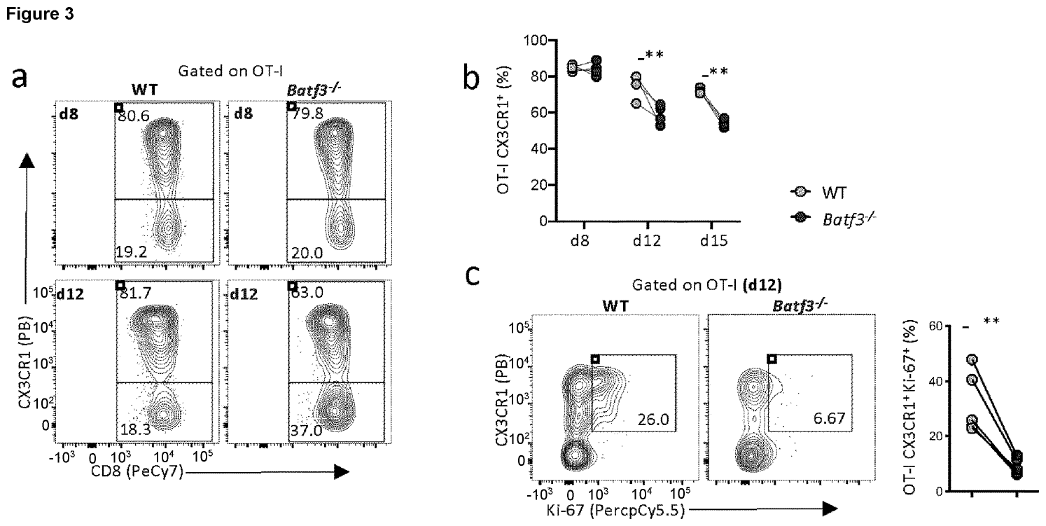

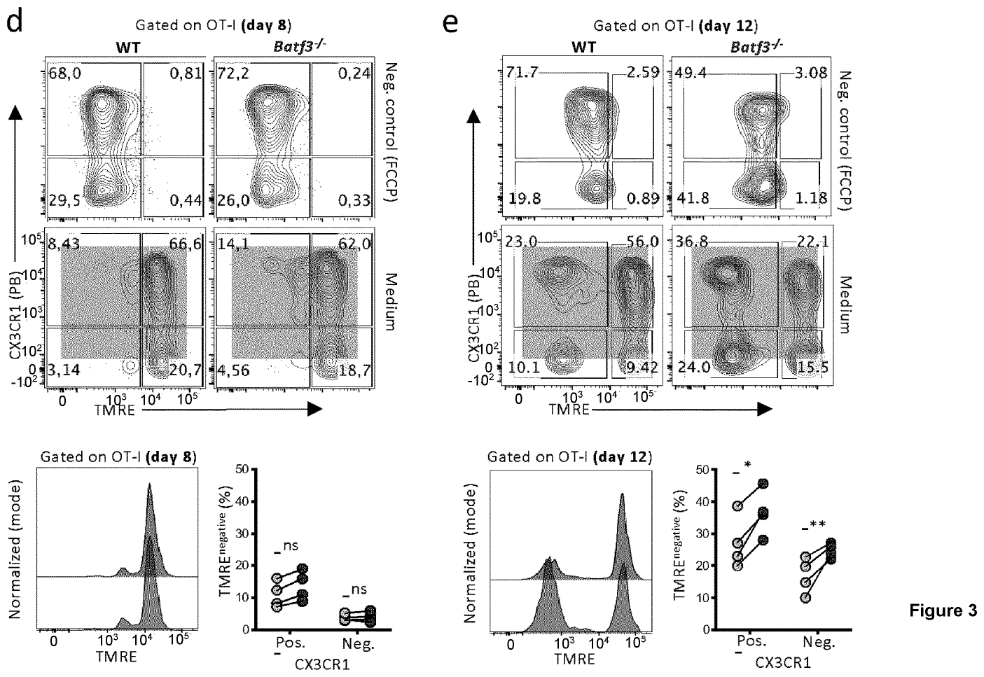

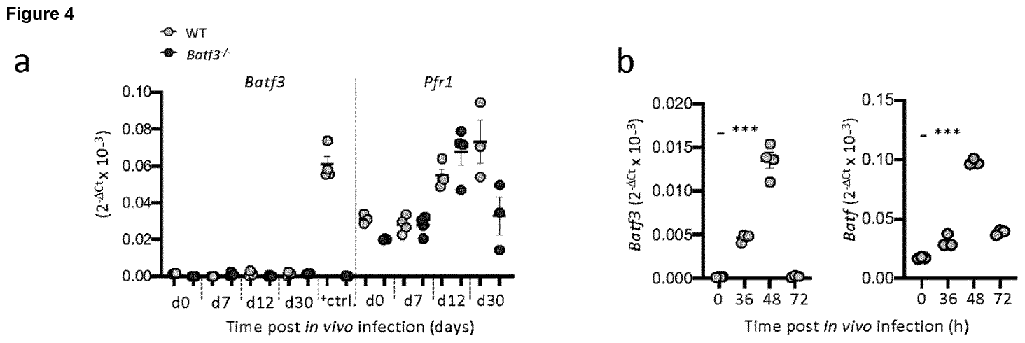

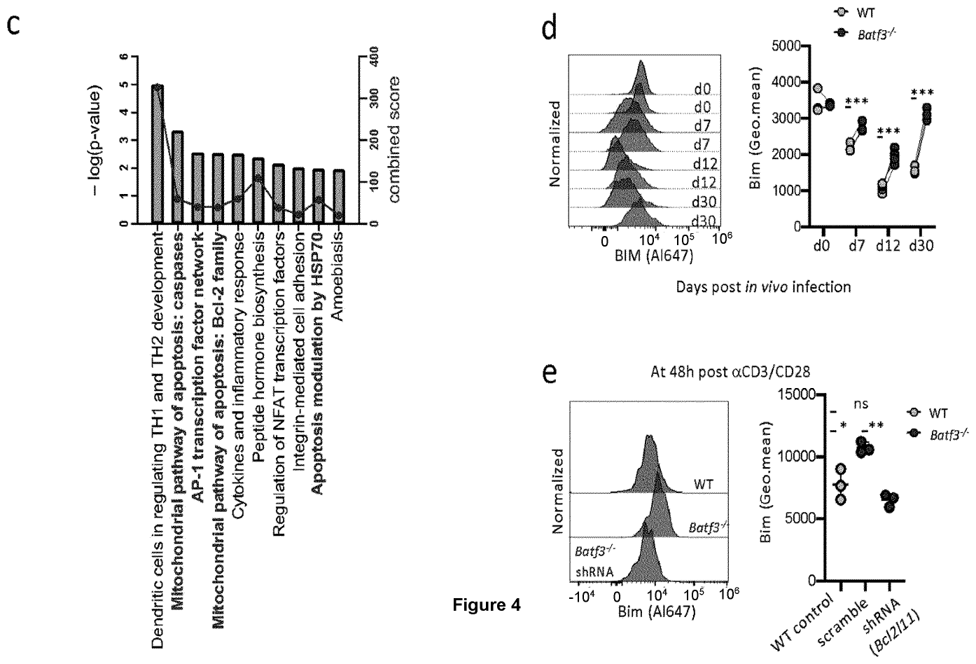

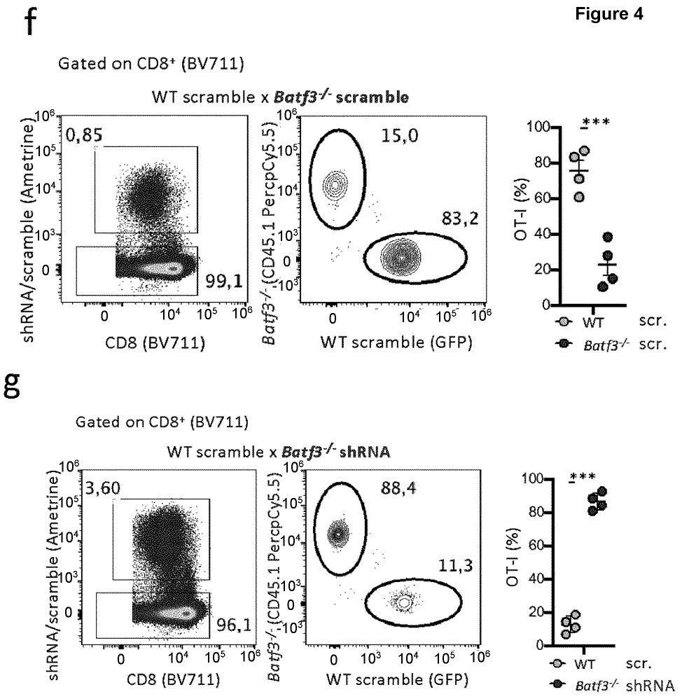

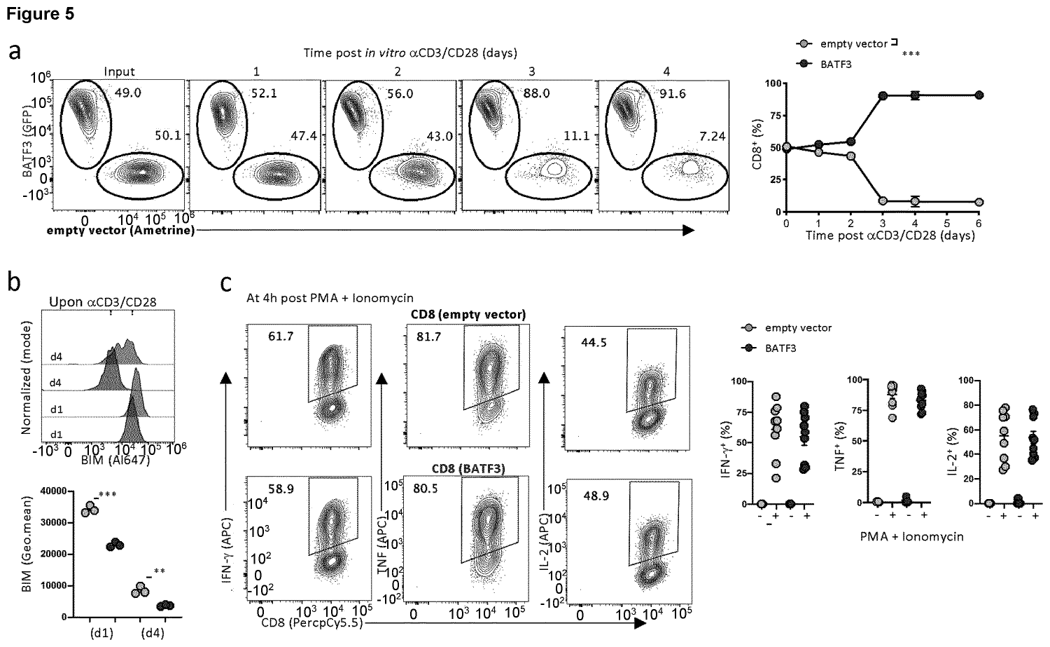

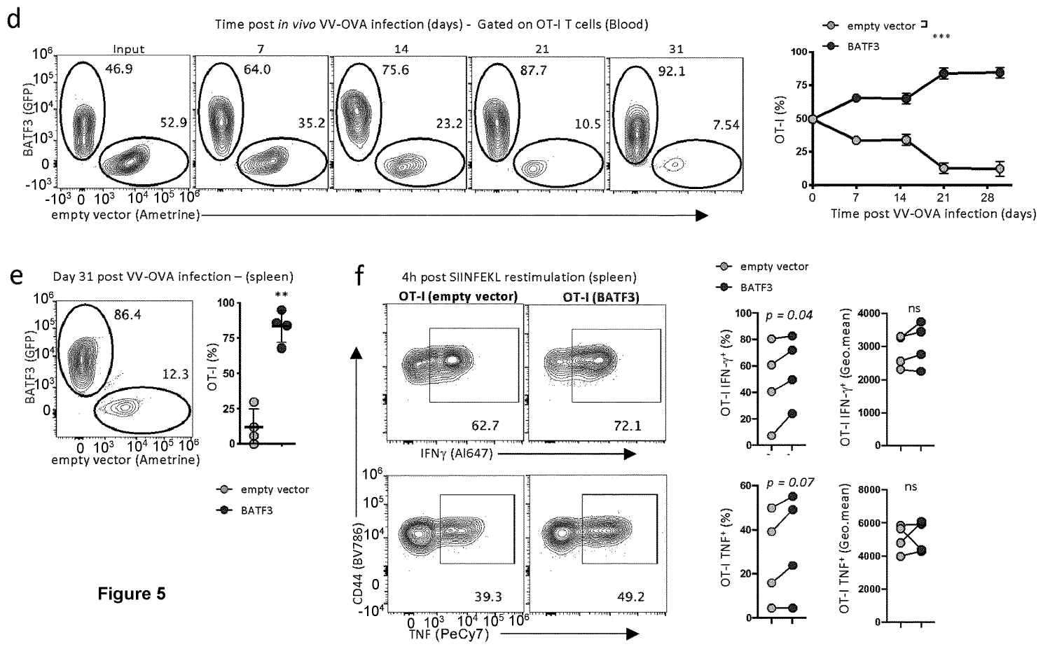

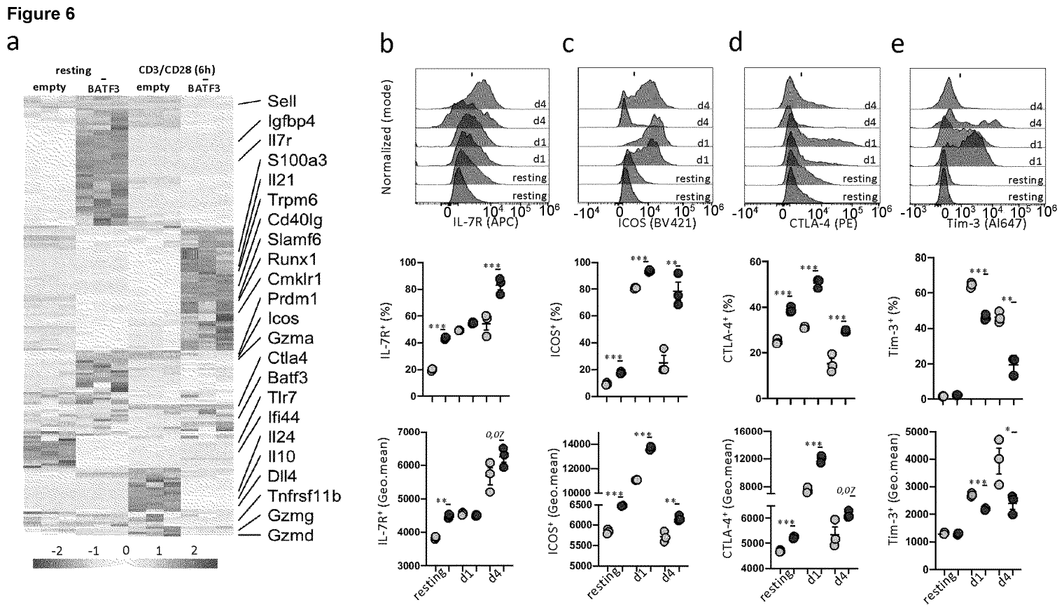

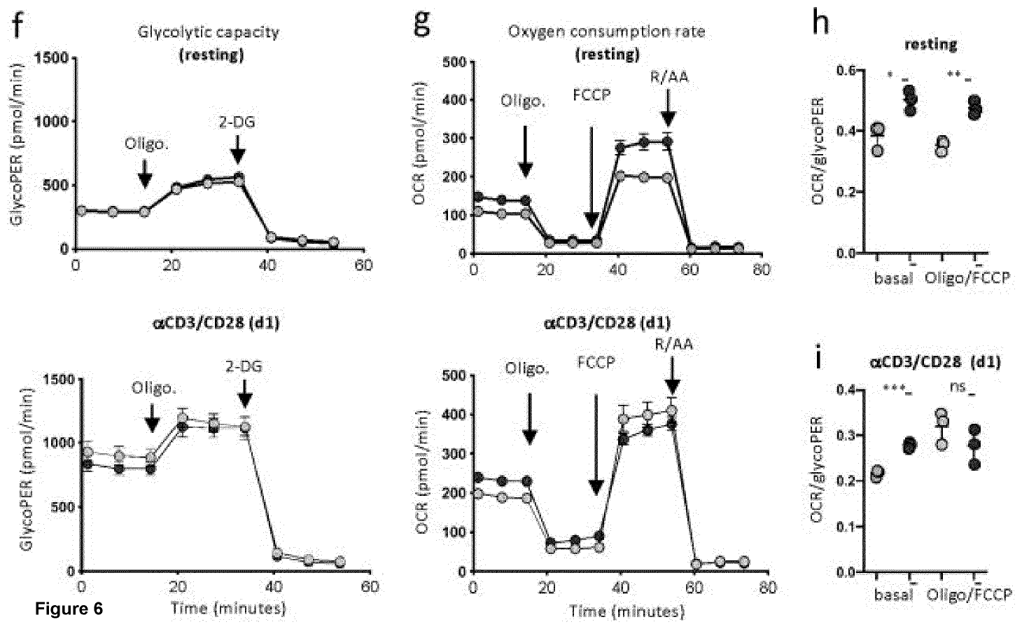

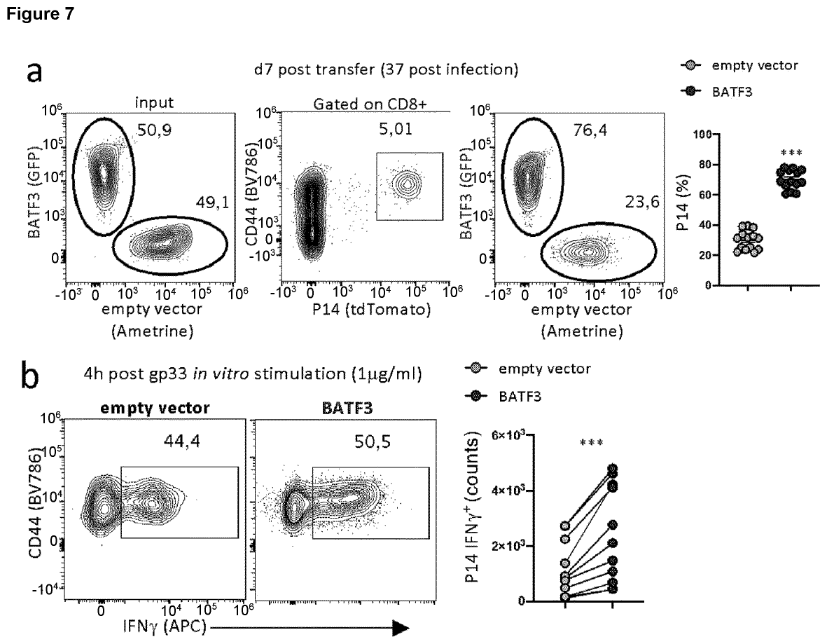

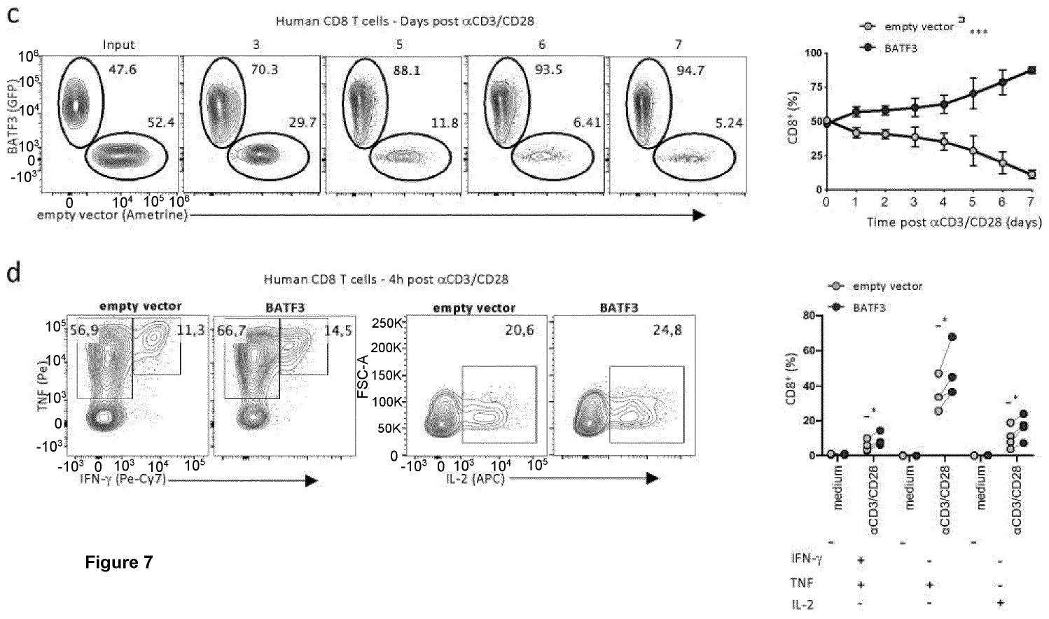

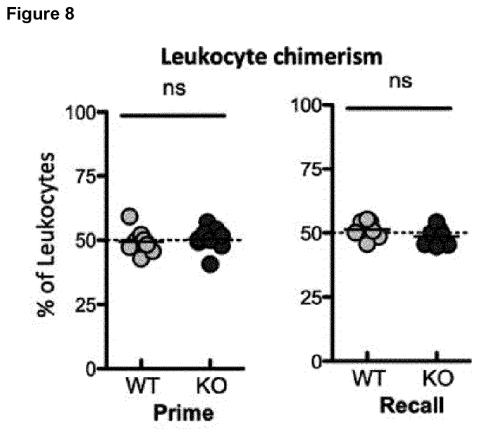

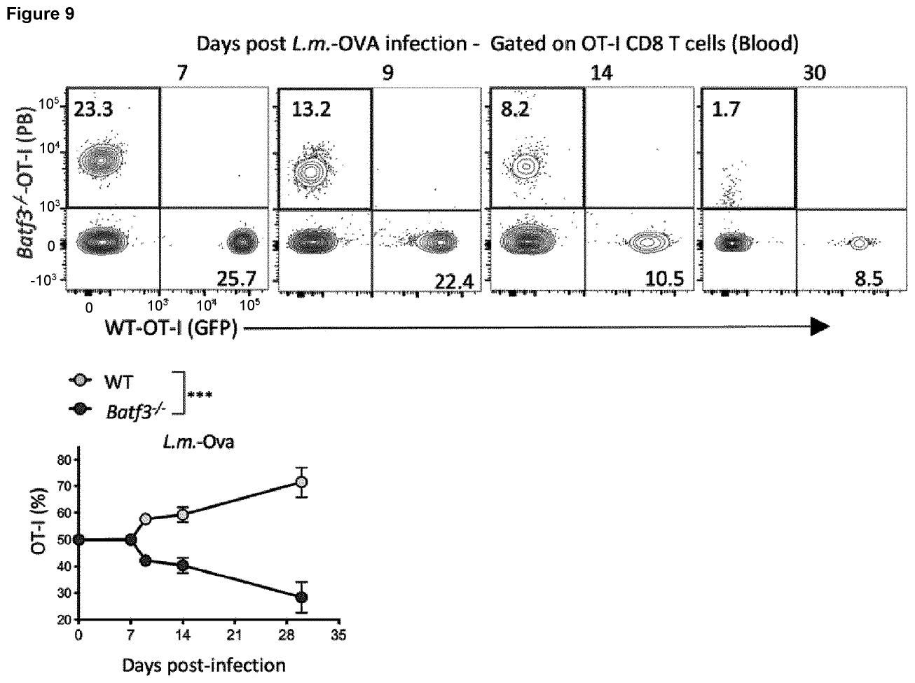

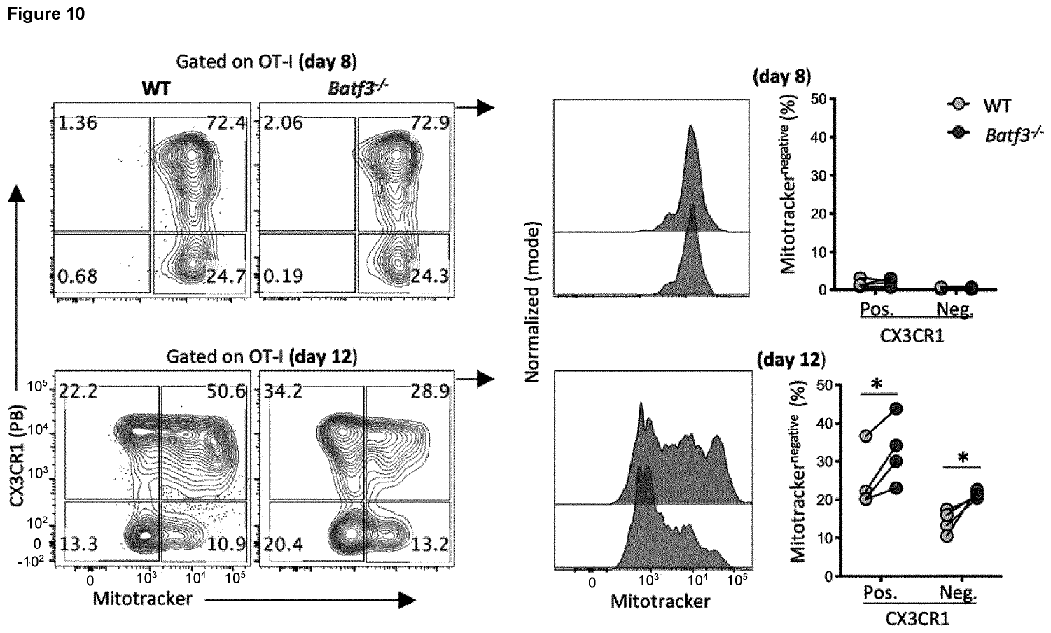

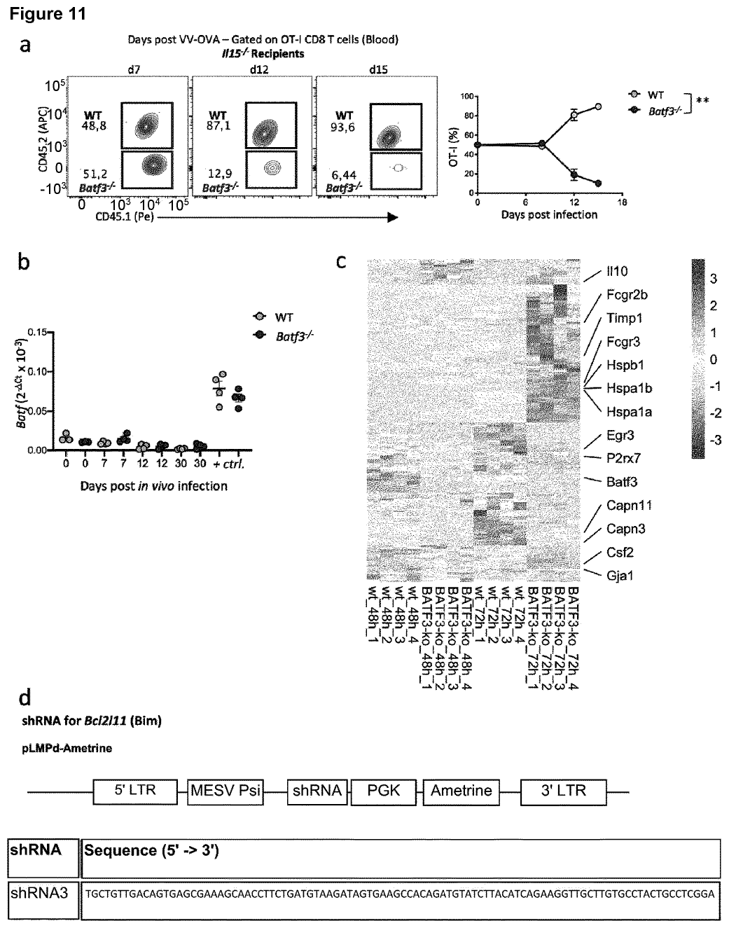

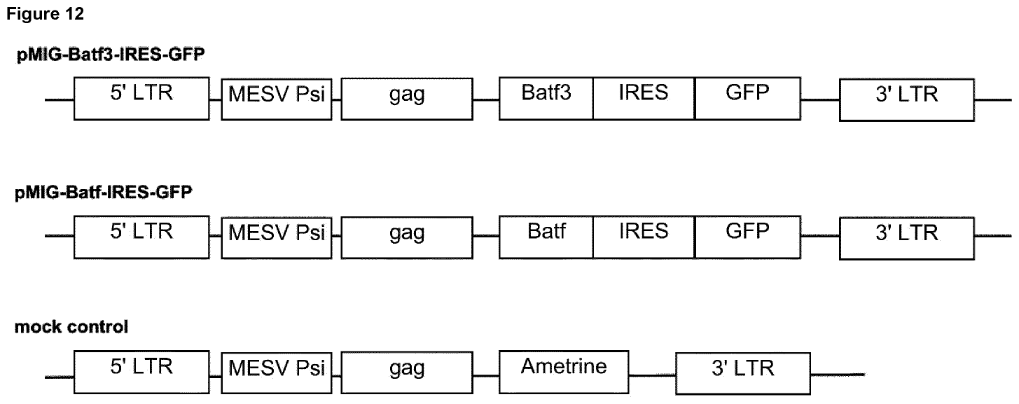

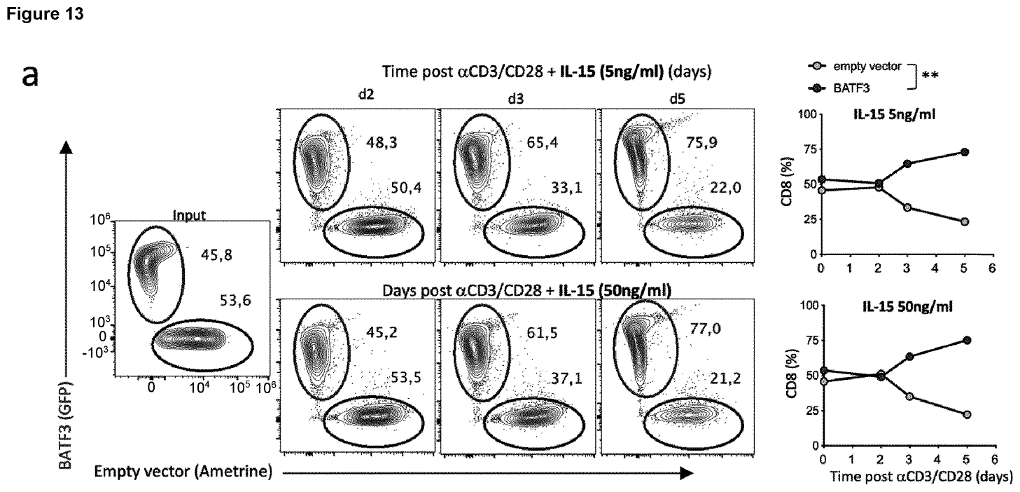

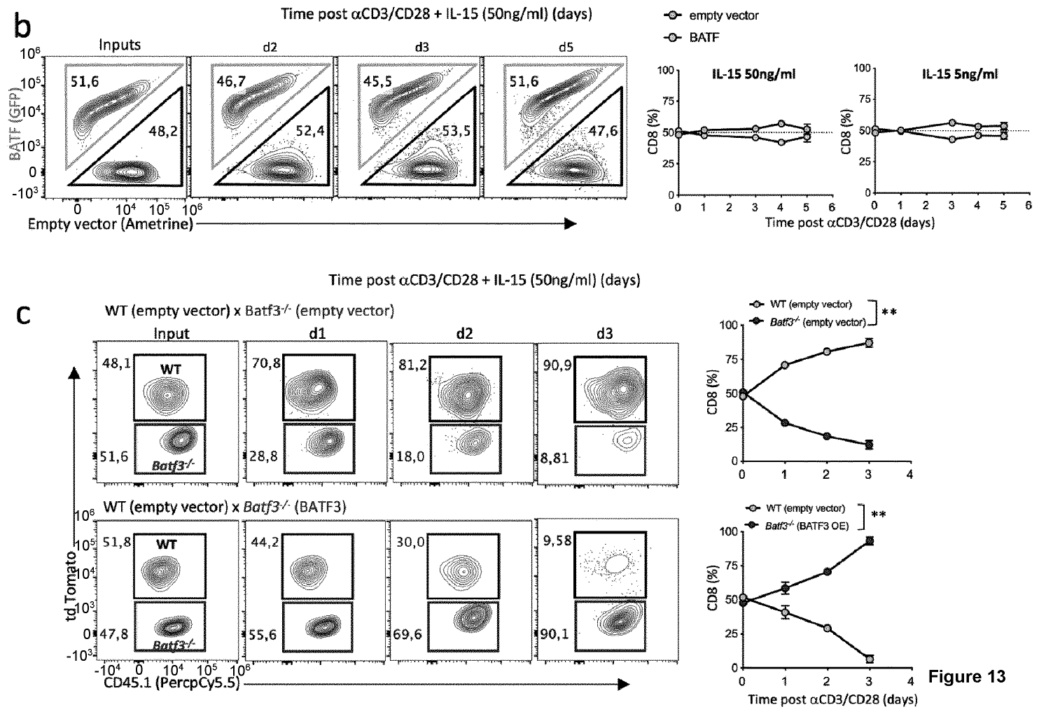

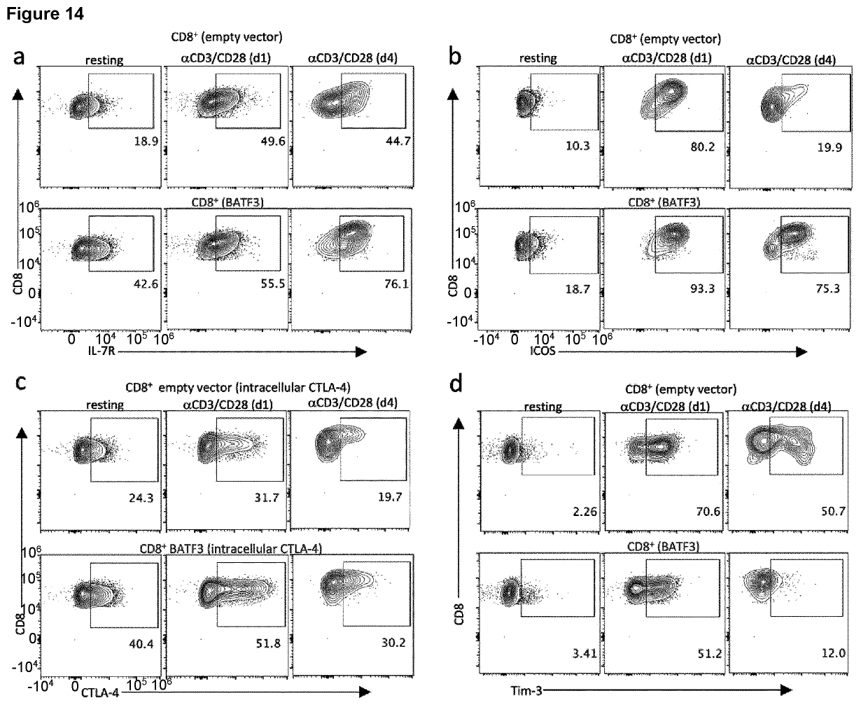

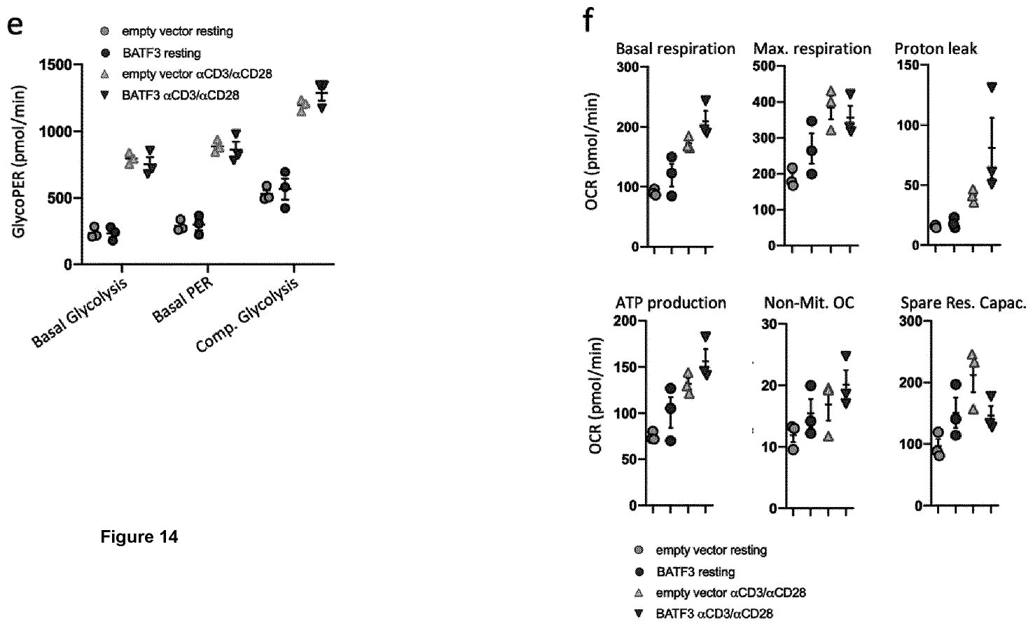

Batf3-deficient mice are widely used to investigate the function of cDC1; however, the inventors speculated that CD8 T cells might also be directly affected by the loss of this transcription factor. Therefore, the inventors wished to delineate the function of BATF3 in CD8 T cell biology. To this end, the inventors infected WT or Batf3^{-/-} mice with Vaccinia virus (VV) i.p. and analyzed the CD8 T cell response directed against the immunodominant epitope B8R₂₀ using flow cytometry on day 8 post primary and day 6 post recall infection (FIG. 1a). There was no significant difference in the B8R-specific CD8 T cell response in Batf3^{-/-} as compared to WT mice (FIG. 1b/c). By contrast, after re-challenging mice with VV on d30 after primary infection the inventors found a striking difference when comparing the B8R-specific immune response between WT and Batf3-deficient animals (FIG. 1b/c). In line with the critical function of cDC1 to mediate CD4 helper signals, Batf3^{-/-} mice showed a substantially impaired CD8 T cell recall response [3, 4]. However, BATF3 is also expressed in CD8 T cells, peaking shortly after activation (48 h) (FIG. 1d) In order to address a possible T cell intrinsic function of BATF3, the inventors generated WT:Batf3^{-/-} mixed bone-marrow (BM) chimeric mice and infected them with VV (FIG. 1e and Supplementary FIG. 1). Probing B8R-specific CD8 T cells in the blood, the inventors detected a similar contribution of WT and Batf3-deficient cells at the primary response against VV on d8 p.i. (FIG. 1f). By contrast, after recall infection B8R-specific CD8 T cells originating from WT cells dominated the overall immune response (FIG. 1f). These results pointed to a T-cell intrinsic function of Batf3 that seemed critical for optimal CD8 T cell memory development or recall capacity. To further characterize the T cell intrinsic role of BATF3, the inventors crossed Batf3^{-/-} mice with TCR-transgenic OT-I mice to perform competitive (50/50) transfer experiments with WT and Batf3-deficient OT-I, allowing for a side-by-side, quantitative and qualitative comparative analysis over time (FIG. 1g). Recipient animals were either infected with VV-OVA or L.m.-OVA (Listeria monocytogenes) and analyzed on d8 p.i. (Prime) or were rechallenged heterologously on d30 and analyzed on d6 post secondary infection (Recall, FIG. 1g). In line with the inventors' previous results, the inventors detected similar amounts of WT and Batf3^{-/-} OT-I T cells in the spleen on d8 after either VV-OVA or L.m.-OVA primary infection (FIG. 1h/i). By contrast, during recall responses Batf3^{-/-} OT-I T cells were largely outcompeted by their WT counterparts (FIG. 1h/i). Despite these quantitative differences, the remaining Batf3-deficient OT-I T cells had a similar capacity to produce effector cytokines, such as IL-2, TNFα and IFNγ, compared to WT cells (FIG. 1j/k). Likewise, the CD8 T cell differentiation based on KLRG1/CD127 expression pattern on d8 after primary infection did not reveal any differences between WT and KO OT-I T cells (FIG. 11). Thus, Batf3-deficient CD8⁺ T cells have normal phenotypical and functional differentiation pattern, despite a defective memory formation.

Having established that Batf3^{-/-} CD8 T cells failed to develop a proper memory response, the inventors next addressed whether this phenotype was based on an impaired transition to memory during the contraction phase or due to a specific defect during recall responses or both. To this end, the inventors performed a kinetic analysis of WT and Batf3^{-/-} OT-I T cells in the blood and the spleens of L.m. and VV infected animals (FIG. 2a/b and FIG. 9. Interestingly, the inventors detected an aggravated contraction of Batf3^{-/-} OT-I T cells as compared to their WT counterparts over time, resulting in significantly reduced numbers of Batf3-deficient OT-I cells by d30 p.i. Next, the inventors analysed the expression levels of CX3CR1 and IL7R on d30 p.i. in order to discern central memory (Tcm), effector memory (Tem) and the recently described peripheral memory CD8 T cell subsets [27, 28]. The inventors found that all memory subsets were reduced in absolute numbers in Batf3-deficient as compared to WT OT-I T cells. Notably, this effect was most dramatic within CX3CR1⁺ Tem indicated by the additional relative loss of this subset (FIG. 2c/d). To complete the analyses on the memory T cell compartment, the inventors generated tissue-resident memory (Trm) CD8 T cells by intradermal infection of the ear. In line with the inventors' previous results the inventors observed a pronounced reduction of Batf3-deficient vs WT OT-I cells using either 2-photon in situ imaging or FACS analyses of tissue homogenates of the skin (FIG. 2e-g). Based on these results the inventors conclude that all memory CD8 T cell subsets are affected by the loss of Batf3.

To test whether the remaining Batf3^{-/-} OT-I memory T cells had a similar capacity to mount recall responses on a per cell level, the inventors isolated splenic memory populations (WT and Batf3^{-/-}) and transferred equal numbers into naïve WT recipients and infected them with VV (FIG. 2h). On d6 post infection the inventors detected a small but significant reduction of Batf3^{-/-} as compared to WT OT-I T cells in the spleens (FIG. 2i). From these results the inventors conclude that Batf3 is critical for both the transition to and the quality of memory CD8 T cells.

Since CX3CR1-expressing Tem cells were severely affected by the loss of Batf3, the inventors further_analyzed CX3CR1 expression on WT and Batf3^{-/-} OT-I T cells between d8 and d15 post infection, a time-frame during which the inventors had observed an enhanced contraction of Batf3-deficient T cells. Consistent with the inventors' previous results, the inventors found the most aggravated loss of Batf3^{-/-} OT-I T cells within the CX3CR1-expressing fraction (FIG. 3 a/b). The overall pronounced loss of Batf3^{-/-} OT-I T cells may be based on a combination of increased cell death, reduced proliferation rates and subset trans-differentiation. Indicative for reduced proliferation rates, the inventors found a significantly smaller fraction of Ki-67 positive cells within Batf3^{-/-} vs WT OT-I T cells at d12 post infection (FIG. 3c). Since apoptotic cells are rapidly removed in vivo, the inventors decided to measure the mitochondrial membrane potential as a proxy for cellular fitness. To this end, the inventors applied two different dyes, a strict staining protocol and parallel measurement of WT vs Batf3^{-/-} OT-I T cells within the same sample on d8 and d12 post infection. On day 8 p.i. the inventors detected no differences between WT and Batf3^{-/-} OT-I T cells when staining for TMRE (FIG. 3d) or mitotracker deep-red (Supplementary FIG. 2). By contrast on d12 p.i. the inventors found a significantly higher fraction of TMRE (FIG. 3e) and mitotracker deep-red (Supplementary FIG. 2) negative cells within the Batf3^{-/-} compared to the WT OT-I T cells, suggesting that Batf3-deficient cells show elevated cell death. This difference was particularly pronounced within CX3CR1-expressing OT-I T cells in line with the most aggravated loss of this subset by d30 post infection.

In order to gain mechanistic insights the inventors first characterized the expression pattern of Batf3 over time using ex vivo isolated TCR-transgenic T cells after VV infection. Reflecting the inventors' in vitro data and similar to the expression pattern of Batf, Batf3 expression peaked early after T cell activation, but was absent during the effector or memory phase (FIG. 4 a/b). Having established the temporal expression pattern of Batf3, the inventors performed an RNAseq analysis comparing WT vs Batf3^{-/-} OT-I T cells 48 h and 72 h post activation (FIG. 11c). Unsupervised analysis revealed an enrichment of mitochondrial pathways of apoptosis (FIG. 4 c). Since BCL2L11 (BIM) is a mitochondrial protein and known proapoptotic factor that regulates CD8 T cell contraction, the inventors investigated the protein levels of BCL2L11 in WT and Batf3^{-/-} OT-I T cells over time. Batf3-deficient and-proficient naive CD8 T cells expressed similar levels of BCL2L11. However, during the course of infection Batf3-deficient CD8 T cells failed to downregulate BCL2L11 and expressed higher levels as compared to WT CD8 T cells (FIG. 4d). Based on this striking correlation the inventors asked whether BCL2L11 is functionally linked to the pronounced contraction seen in Batf3-deficient CD8 T cells. To this end, the inventors aimed at normalizing BCL2L11 gene expression using shRNA mediated knock down (FIG. 4e and FIG. 11d). Next, the inventors transduced WT and Batf3-deficient OT-I T cells with control shRNA or shRNA targeting BCL2L11, transferred them into mice at a 50/50 ratio (FIG. 4f). On d7 post infection the inventors found 10-fold more WT than Batf3-deficient OT-I T cells that were transduced control shRNA, as expected (FIG. 4g/h). By contrast Batf3-deficient OT-I T cells transduced with retrovirus expressing shRNA against BCL2L11 outcompeted WT T cells that expressed control shRNA (FIG. 4g/h). Therefore, the inventors conclude that the pronounced T cell contraction and impaired memory CD8 T cell formation observed in the absence of BATF3 is at least in part based on a dysregulation of BCL2L11. Notably, WT vs Batf3^{-/-} OT-I T cells transferred into IL15-deficient recipients showed a similar difference as in WT recipients arguing against a critical role for IL15 signals in mediating the effects seen in Batf3^{-/-} CD8 T cells (FIG. 11a and FIG. 13a).

Having established that Batf3-deficiency leads to impaired CD8 T cell memory the inventors asked whether vice versa gain-of-function of BATF3 could further improve CD8 T cell memory generation. To this end, the inventors transduced naïve CD8 T cells retrovirally with a BATF3-overexpressing vector (pMIG expression BATF3-IRES-GFP) or an empty control plasmid (pMIG expressing Ametrine only) (FIG. 5a and FIG. 12). When culturing these cells at a 50/50 ratio in vitro, the inventors found that BATF3-overexpressing CD8 T cells almost completely outcompeted their empty vector controls over time after restimulation with anti-CD3/CD28 (FIG. 5b). In line with the inventors' previous results, the inventors detected significantly lower levels of the proapoptotic molecule BCL2L11 within BATF3-overexpressing cells, suggesting resistance to apoptosis (FIG. 5c). Similar as in Batf3-deficient cells, BATF3-overexpression did not perturb the production of cytokines, such as IFNγ, TNFα and IL2 (FIG. 5d). Next the inventors wished to address whether BATF3-overexpression also endows CD8 T cells with a superior capacity to survive in vivo. Therefore, the inventors co-transferred BATF3-overexpressing and empty vector control OT-I T cells into naïve mice and infected them with VV-OVA (FIG. 5e). By d7 p.i. BATF3-overexpressing GFP⁺ OT-I cells already outcompeted their ametrine⁺ OT-I control cells in the blood (FIG. 5e). After 31 days, the memory pool of OT-I cells was almost exclusively composed of BATF3-overexpressing T cells in both blood and spleens of previously infected mice (FIG. 5f). After restimulating splenocytes ex vivo, the inventors found that the BATF3-overexpressing OT-I cells had a slightly higher capacity to produce IFNγ and a trend to produce more TNFα than their empty vector controls (FIG. 5g). Having established that both loss- and gain-of-function experiments indicate a critical role of BATF3 in regulating CD8 T cell fitness and memory, the inventors next applied RNAseq in order to gain an unbiased view on the molecular changes induced by BATF3. Specifically, the inventors compared the transcriptome of resting or anti-CD3/CD28 restimulated OT-I T cells that were transduced with either BATF3-overexpressing or control vectors (FIG. 6a). The inventors detected more than 180 differentially regulated genes that fell into six different categories (FIG. 6b). The first category were genes that were upregulated in BATF3-overexpressing cells during the resting phase. These genes may play a particular role during transition and/or maintainence of memory CD8 T cells. Among them were genes such as 117r, Sell and Igfbp4 with known functions in lymphocyte survival, recirculation capacity and metabolism. The second category included genes that were particularly upregulated upon restimulation of BATF3-overexpressing cells. This group included genes important during the effector phase of CD8 T cells, such as the cytokine II21, the transcription factor Runx1, the costimulatory molecule Cd40Ig, and the magnesium transporter Trpm6. The third category involved genes that were upregulated during both the resting and the reactivation time-point and included the transcription factor Prdm1, the costimulatory molecules Ctla4 and Icos and the effector molecule Gzma. Category 4 included genes that were downregulated upon BATF3-overexpression in the resting phase like Tlr7 and Ifi44—genes that are associated with inflammation. Category 5 contained genes that were downregulated in BATF3-overexpressing cells during reactivation and included genes for anti-inflammtory cytokines like 1110 and 1124. Finally, within category six were genes that are downregulated during both the resting and the reactivation phase for instance the proteases Gzmd and Gzmg. In summary, BATF3-overexpression upregulates multiple genes that are connected to CD8 T cell memory, while it downregulates anti-inflammatory mediators and sensors of inflammation. Next the inventors wished to confirm the detected mRNA differences on a protein level for some selected genes with a known function in T cell biology. To this end, the inventors performed FACS analysis on BATF3-transduced polyclonal CD8 T cells before and after re-stimulation with anti-CD3/CD28. The inventors found a significantly higher fraction that expressed IL7R within the BATF3-overexpressing vs empty vector transduced CD8 T cells at resting and d4 time-points (FIG. 6c and FIG. 14a). Additionally, among IL7R positive cells, BATF3-overexpressing cells had a higher mean fluorescence intensity of αIL7R staining, indicating higher per cell levels of IL7R than their control counterparts. Similarly striking results were obtained for the costimulatory molecule ICOS, which was highly upregulated within BATF3-overexpressing cells upon restimulation (FIG. 6d and FIG. 14b). Next, the inventors tested the expression of coinhibitory molecules. As indicated by the RNAseq data, the inventors confirmed higher levels of CTLA-4 in BATF3-overexpressing cells in the resting state and after reactivation using intracellular staining (FIG. 6e and FIG. 14c). By contrast another coinhibitory molecule Tim-3 was significantly downregulated upon cell activation in the context of BATF3-overexpression (FIG. 6f and FIG. 14d).

CD8 T cell longevity and memory is further characterized by an altered metabolism compared to (short-lived) effector T cells [29]. Quiescent and memory T cells utilize preferentially oxidative mitochondrial metabolism and fatty acid oxidation and show relatively little glycolytic activity to fuel their bioenergetic demand [30, 31]. To better understand how BATF3 promotes the memory phenotype in CD8 T cells, the inventors analyzed aerobic glycolysis and oxidative phosphorylation (OXPHOS) in BATF3 overexpression and control cells before and after anti-CD3/CD28 re-stimulation using a seahorse flux analyzer (FIG. 6g-j and FIG. 14e/f). The inventors first measured the proton efflux rate (glycoPER), which indicates lactate production by aerobic glycolysis, under basal conditions and after inhibition of mitochondrial metabolism following oligomycin treatment (FIG. 6g and FIG. 14e/f). In comparison to resting cells, which showed a lower basal and maximal glycolytic activity, restimulation with anti-CD3/CD28 induced a significant increase in extracellular acidification in both BATF3 expressing and control cells (FIG. 6g and FIG. 14e/f). Similarly, the oxygen consumption rate (OCR), as a measure for OXPHOS, was elevated after re-stimulation with anti-CD3/CD28, albeit to a lesser extend compared to the marked upregulation of glycoPER (FIG. 6h and FIG. 14e/f). Under basal conditions (before oligomycin treatment) the inventors noticed a small but reproducible increase of OCR in BATF3 transduced CD8 T cells compared to control cells (FIG. 6h), suggesting that BATF3 promoted elevated mitochondrial metabolism. The ratio of OCR to aerobic glycolysis (glycoPER) is a useful measure to describe the metabolic phenotype of T cells independent of their overall metabolic activity [31]. Importantly, the inventors observed significantly higher OCR:glycoPER ratios in BATF3 overexpressing T cells compared to control cells both under resting conditions (FIG. 6i) and after restimulation with anti-CD3/CD28 (FIG. 6j), demonstrating that BATF3 promotes a metabolic program that is highly reminiscent of memory CD8 T cells.

Together, these data argue that BATF3-overexpression induces complex changes that overall promote T cell survival and metabolic fitness. Based on the inventors' observation that the expression pattern of coinhibitory and costimulatory molecules is also controlled by BATF3, the inventors speculated that ectopic BATF3-expression in CD8 T cells may also provide an advantage in the context of persistent antigenic stimulation as during chronic viral infections. To test this notion, the inventors transduced P14 CD8 T cells with BATF3-overexpression or control vectors and transferred them into mice that were chronically infected with LCMV C113 (FIG. 7a). As anticipated, the inventors detected significantly more BATF3-overexpressing compared to control cells one week post transfer in the spleens of chronically infected mice (FIG. 7b). Both transferred cell populations were similarly able to produce inflammatory cytokines, yet the absolute number of IFNγ producing was significantly higher within the BATF3-overexpressing population (FIG. 7c).

So far, the inventors showed that BATF3 promotes cellular fitness and memory formation in murine models after viral infection, but if BATF3 has a similar role in human CD8 T cells is not known. In order to test if the pro-survival function of BATF3 is conserved, the inventors transduced human CD8 T cells from healthy donors with BATF3 and empty vector expression plasmids similar to the inventors' experiments with murine T cells. FACS-sorted BATF3-overexpressing and control CD8 T cells were co-cultured and restimulated with anti-CD3/CD28 beads for up to 7 days. Similar to murine CD8 T cells, human CD8 T cells overexpressing BATF3 also outcompeted their control cells over time (FIG. 7d) demonstrating that BATF3 has a conserved role across species to promote T cell longevity. When analysing the cytokine production, the inventors detected a small but consistent increase in the capacity of BATF3-overexpressing human CD8 T cells to produce inflammatory cytokines like IFNγ, TNFα and IL2 (FIG. 7e). From this data the inventors conclude that BATF3-overexpression leads to increased amounts of CD8 T cells in vitro upon activation while fully preserving their functional capacity to produce cytokines. Together these results warrant further investigation of the therapeutic potential of BATF3 for adoptive immunotherapy in humans.

### DISCUSSION

Here, the inventors identified a physiological, cell-intrinsic function of BATF3 in regulating CD8 T cell survival, proliferation and their transition to memory. Batf3-deficient mice are a widely used model to study the role of cDC1 in the context of tumors and infections. The inventors' study does not question the well-established role of cDC1 in cross-presenting antigen, driving antiviral immunity and serving as a critical platform to mediate CD4 helper signals. However, results obtained with Batf3-deficient mice should be carefully interpreted under the light of the inventors' work. Future studies aiming to address the role of cDC1 should apply cell-specific models that are based on e.g. Xcr1 [32, 33, 34, 35].

In the inventors' study the inventors found that in CD8 T cells the basic leucine zipper protein BATF3 is transiently expressed early after T cell activation but has long lasting effects on T cell contraction, cellular/mitochondrial fitness, longevity and therefore on CD8 T cell memory. Both intrinsic and extrinsic factors regulate T cell contraction and longevity [36]. Extrinsic factors are homeostatic cytokines like IL7 and IL15 or inflammatory cytokines like IL12, IFN I and IFNγ. These factors don't seem to be involved in regulating the aggravated contraction seen in the context of Batf3-deficiency. Well-established intrinsic factors of T cell contraction are the transcription factors Tbet, eomesodermin, Id2 and BCL6. While these factors share a function with BATF3 to regulate CD8 T cell survival, they differ with regards to their impact on T cell differentiation. Transcription factors that regulate the balance of effector (KLRG1lo CD127hi) vs memory T cell (KLRG1hi CD127lo) development naturally impact on the overall size of the long-term T cell response. By contrast, BATF3-deficiency did not alter CD8 T cell differentiation but regulated the survival of the entire CD8 T cell population, notably most pronounced CX3CR1-expressing Tem cells. The critical downstream effector molecule of BATF3 is BCL2L11 a central regulator of apoptosis and T cell contraction [37, 38]. BATF3 has been shown to bind to the BCL2L11 promoter region and as a transcriptional repressor could negatively regulate BCL2L11 expression [17]. Consistently, the inventors were able to rescue Batf3-deficiency in vivo by knockdown of BCL2L11. Since BCL2L11 is dynamically regulated over time it is currently not possible to fully normalize BCL2L11 in Batf3-deficient OT-I T cells to physiological levels seen in WT OT-I T cells. Therefore, the fact that Batf3-deficient CD8 T cells with a knock down of BCL2L11 outcompeted WT CD8 T cells in vivo likely reflects a reduction of BCL2L11 below WT levels. BCL2L11 expression is regulated on multiple levels and is influenced by a whole range of transcription factors and epigenetic modifiers [39, 40]. Notably, while the inventors detected clear changes in BCL2L11 protein levels in settings of loss- or gain-of-function of BATF3 in CD8 T cells, the inventors did not detect corresponding changes on BcI2I11 mRNA levels. Similarly BcI2I11 mRNA levels did not correlate with BCL2L11 protein levels in WT OT-I T cells over the course of infection (data not shown). In vivo the dysregulation of BCL2L11 in Batf3-deficient CD8 T cells becomes apparent after the physiological expression of BATF3 in WT CD8 T cells has ceased. This argues for epigenetic changes that are imprinted by BATF3 within the first days of T cell activation with long-lasting effects on cell survival and proliferation, which are indeed key functions of AP-1 [41]. Alternatively, there may be temporal spikes of BATF3 expression and/or within a fraction of contracting antigen-specific CD8 T cells that are below the inventors' detection limit. The role of BATF3 to imprint T cell memory early after activation is reminiscent of the role of CD4 help for CD8 T cells that is transmitted during the same time frame after activation (24-72 h). Also the phenotypic parallels between lack of BATF3 and lack of CD4 help are remarkable and it is intriguing to speculate that at least in part CD4 helper signals may be executed via the same network of transcriptions factors [42]. Within dendritic cells, BATF3 cooperates with IRF8 to execute its transcriptional activity [13]. Since IRF8 is also expressed in CD8 T cells upon activation it appeared to be a likely co-factor for BATF3 within T cells [43]. In the inventors' hands, however, the inventors did not detect any differences between WT and Irf8-deficient OT-I T cell after primary or secondary responses with VV-OVA (data not shown). Therefore, the key binding partner of BATF3 to mediate its effects in CD8 T cells remains unanswered.

The inventors' study has revealed a physiological function of BATF3 in CD8 T cells by regulating memory development and long-term survival. Vice versa, the inventors were able to demonstrate that BATF3 overexpression further enhanced T cell persistence in vivo making it a suitable target to optimize adoptive T cell therapy approaches. As seen in Batf3-deficient CD8 T cells, BATF3-overexpression also induced complex context-dependent transcriptional changes. Only a small number of BATF3 target genes were up- or downregulated both in the resting state and after re-stimulation with anti-CD3/CD28, including CTLA-4 a known target of BATF3 transcriptional activity [17]. By contrast the majority of target genes were modulated in a context dependent manner—either in the resting state or after activation. Consistent with the observed enhanced expression of BCL2L11 in 8c/0-deficient CD8 T cells leading to a pronounced contraction, BATF3-overexpression led to reduced BCL2L11 levels supporting its role as a critical downstream molecule of BATF3.

A recent study that screened for factors that regulate T cell exhaustion in the context of cancer identified REGNASE-1 [44]. Regnase-1-deficient CD8 T cells had an enhanced capacity to eliminate tumor cells in vivo. Notably, the authors showed that Regnase-1 deficiency increased BATF protein levels and that BATF-overexpression in turn induced metabolic changes and increased the number of tumor infiltrating CD8 T cells in vivo. These data are consistent with the impact of BATF3 overexpression on CD8 T cell abundance observed in the inventors' study. Indeed, the inventors found that BATF overexpression also lead to reduced levels of BCL2L11 in CD8 T cells arguing for a similar mechanism (data not shown).

Another study that investigated the molecular basis of exhaustion of CAR T cells identified a dysbalance in the AP1-IRF network as an underlying mechanism of exhaustion [45]. The authors found that the overexpression of cJUN could rebalance these factors likely by forming additional cJUN/FOS dimers. Additionally, cJUN will likely form heterodimers with BATF and BATF3 thereby influencing interactions between BATF/BATF3 and IRF family members. As a consequence, cJUN-overexpressing CAR T cells remained functional and acquired a resistance towards exhaustion. Supporting this notion cFOS-overexpression, another AP1 factor that does not bind to BATF family members could not revert exhaustion. Putting these and the inventors' studies together the inventors conclude that both reduced and increased levels of BATF can enhance the anti-tumor activity of CD8 T cells depending on the context. Therefore, while the AP1-IRF family network seems to be a suitable target to reinvigorate exhausted CD8 T cells, the details how to modulate this network or how it is changed upon modulating single elements requires further investigation. The fact that BATF family members cooperate with IRF's (transcriptional activation) and multiple other AP1 family members like cJUN (transcriptional repression) and that all these factors are dynamically regulated during T cell activation poses a significant challenge in delineating how quantitative changes of these molecules contribute to the overall outcome of the network and ultimately CD8 T cell fitness. A general caveat of modulating the AP1-IRF network is the potential induction of malignant transformation [46]. Indeed, transgenic mice overexpressing human BATF showed the development of lymphoproliferative disease [25]. Similarly, overexpression of human BATF3 in murine lymphocytes causes B cell lymphomas in mice [47]. Although the induction of T cell lymphomas was not observed, these findings underscore the need to introduce a safety switch in genetically modified adoptively transferred T cells, which is already commonly used to limit off-target toxicity.

In summary, the inventors identify a physiological role of BATF3 in regulating CD8 T cell memory by modulating the proapoptotic factor BCL2L11. The inventors further demonstrate that this function of BATF3 can be harnessed to improve T cell survival and longevity in vivo. Given its function in promoting CD8 T cell memory in both murine and human CD8 T cells, BATF3 is a highly suitable candidate for the development of optimized adoptive T cell therapies against cancer. These data have a direct implication for and the invention can be used in the context of adoptive cellular immunotherapy with gene-engineered T cells such as CAR-modified T cells and TCR-transgenic T cells, and non-gene-engineered T cells. Based on these results a similar function of BATF3 in ILC seems very likely. Indeed, our preliminary results in BATF3-deficient NK cells show a similar defect in survival and longevity as seen in BATF3-deficient CD8 T cells.

### INDUSTRIAL APPLICABILITY

The lymphocytes according to the invention can be industrially manufactured and sold as products for the methods and uses as described (e.g. for treating a cancer as defined herein), in accordance with known standards for the manufacture of pharmaceutical products. Accordingly, the present invention is industrially applicable.

Sequences

SEQ ID NO: 1 (peptide)
SIINFEKL
SEQ ID NO: 2 (human BATF3 protein sequence; Q9NR55)
MSQGLPAAGSVLQRSVAAPGNQPQPQPQQQSPEDDDRKVRRREKNRVAAQRSRKKQTQKADKLHEE
YESLEQENTMLRREIGKLTEELKHLTEALKEHEKMCPLLLCPMNFVPVPPRPDPVAGCLPR
SEQ ID NO: 3 (mouse BATF3 protein sequence; Q9D275)
MSQGPPAVSVLQRSVDAPGNQPQSPKDDDRKVRRREKNRVAAQRSRKKQTQKADKLHEEHESLEQEN
SVLRREISKLKEELRHLSEVLKEHEKMCPLLLCPMNFVQLRSDPVASCLPR
SEQ ID NO: 4 (DNA encoding BCL2L11 shRNA(3))
TGCTGTTGACAGTGAGCGAAAGCAACCTTCTGATGTAAGATAGTGAAGCCACAGATGTATCTTACAT
CAGAAGGTTGCTTGTGCCTACTGCCTCGGA
SEQ ID NO: 5 (DNA encoding BCL2L11 shRNA(1))
TGCTGTTGACAGTGAGCGCAGCAACCTTCTGATGTAAGTTTAGTGAAGCCACAGATGTAAACTTACA
TCAGAAGGTTGCTTTGCCTACTGCCTCGGA
SEQ ID NO: 6 (DNA encoding BCL2L11 shRNA(2))
TGCTGTTGACAGTGAGCGAAGTTGTGACAAGTCAACACAATAGTGAAGCCACAGATGTATTGTGTTG
ACTTGTCACAACTCTGCCTACTGCCTCGGA
SEQ ID NO: 7 (human BCL2L11 mRNA transcript variant 1; NM_138621.5; CDS nucleotides
289-885, start and stop codon underlined)
ACUUCGCUCCGCGCAGCCGCCUGGUCUGCAGUUUGUUGGAGCUCUGCGUCCAGCGCCGCUGCC
GCUGCCGCCGCCGCCGCCGCCGCCGCCGCCGCCGCCGCCGCCGCCACUACCACCACUUGAUUCUU
GCAGCCACCCUGCGAACCCUGCCACACUGCGAUCGCAUCAUCGCGGUAUUCGGUUCGCUGCGUU
CCCGCCGCCACCGCCUCGGCGCCCUUUCUUGGCCCUUGUUCCCCCAAAUGUCUGACUCUGACUC
UCGGACUGAGAAACGCAAGAAAAAAAGACCAAAUGGCAAAGCAACCUUCUGAUGUAAGUUCUG
AGUGUGACCGAGAAGGUAGACAAUUGCAGCCUGCGGAGAGGCCUCCCCAGCUCAGACCUGGGG
CCCCUACCUCCCUACAGACAGAGCCACAAGGUAAUCCUGAAGGCAAUCACGGAGGUGAAGGGGA
CAGCUGCCCCCACGGCAGCCCUCAGGGCCCGCUGGCCCCACCUGCCAGCCCUGGCCCUUUUGCUA
CCAGAUCCCCGCUUUUCAUCUUUAUGAGAAGAUCCUCCCUGCUGUCUCGAUCCUCCAGUGGGU
AUUUCUCUUUUGACACAGACAGGAGCCCAGCACCCAUGAGUUGUGACAAAUCAACACAAACCCC
AAGUCCUCCUUGCCAGGCCUUCAACCACUAUCUCAGUGCAAUGGCUUCCAUGAGGCAGGCUGAA
CCUGCAGAUAUGCGCCCAGAGAUAUGGAUCGCCCAAGAGUUGCGGCGUAUUGGAGACGAGUUU
AACGCUUACUAUGCAAGGAGGGUAUUUUUGAAUAAUUACCAAGCAGCCGAAGACCACCCACGA
AUGGUUAUCUUACGACUGUUACGUUACAUUGUCCGCCUGGUGUGGAGAAUGCAUUGACAGGU
UCUUUGCGGAGCCGAGAUACCAUGCAGACAUUUUGCUUGUUCAAACCAACAAGACCCAGCACCG
CGGUCUCCUGGUGCCAUUAUUAUGCAGCCAGCGGUUCUCUUGUGGAGGGGGCAGGUGACGUU
UCAGAAGACACCGAGCUGGAUGGGACUACCUUUCUGUUCAUCACCACACAGCAGAAUUUCUAA
UGGAAGUUUGUUGUGAAUGUAAAGGAGGGAGCAUUCUUUGCUUUUUAAUAUACAAACCAUG
GUUUUUUGGAGCAGGAUUUUGUGUAAGAAUGGUGUUUACAUGCAGUGUGUUUUCCCCCUCA
CCUUCAAUAAGGUUUUUCAAAAAGGAAAUGGAAACUUUUUAACCAAUUUGUGAAUAACUUUU
GUAUUAAAAUUUUAAGAACCUACGGCCUAUUCUCAGAGGAUUAUGUAACCCCUGCAGUGGAAA
CUGAGCCAGCUAACUUAAAAAGCUGCCUUAGUUUAUUUUUAGAGAUUACAGAAUUUUUAAAC
AGGGAGACGUGUGAUAUACUCCCUCCCUUCCCUACUAUUGCCUCUCUGACCUUUUUAAAUUAU
UUUUAAUACCAAAAGAGUUCUUUUGAAAUGGAACUGAUUAAAAGGGCAGAGGGUCUGUUGCC
AGCCUGCAUUGAUAUACCAGUCCCAUUUGUAAAUAUUUACGUACCUUUAUAAAUUCAGUUGCA
UCUGUGGCAAAAUUUCAGACUAUUUUUGCGUCUUUCCUCAUCACUUUUUGUGAUGCAACUCC
AGUCUGGACUCAGAUGCAUAGAUUUGGUCCAGUGUAUUUUCAUGAUAAAGUGAAAUUGAGUC
AGAACAAGAGUUAAUAUCUGCCUGUAUCUUGCACAGUUCGAGCGAUCUGUUAUUAACUGGGA
AGCAUUUGGUGUUGGUUUUCAUUCCAUUUCGACGAGCAUGUUAUUGGGAAGUAUUCUGAAG
AGGCAAUAGCAGUAAUAACAACAGACUUAAGUGCUACGCCCCUUUGUGCUGCUGGCUUUUCUG
GUUGCAGGCUUUCCCAUGGUCACAGGAUGCACUGUCAGCAUCAGGUCCCAGAGGGCCACCGUG
UCCAUUACAGCAGAGUCCAGCUGCAGCAUCCAGCUCACGCCCUCAUGGGAAUUGGCACAGGCCU
GGGGCAGGGCUUCUGAUGGCCAUUUGCUUGGCCUCCUGCAUUUUAGUCCAACUCACAGUCCAC
UAGCUUCACUCCUUUAAAUUCACUUUGAAACAGGCCUCAUCCCACUUCCACCAGCACCAUAGAA
GAAUAAUUCUGGGCAGAAGUCUGUUUUUUUUCAUUUUUCCAGGACAGUUGGAUAUUGUCAG
GCCACUUGUGACCCCAGCCAUGUAGUGAGGGUGCUCUUUCUCUGUGCCUGCUCCUUAUGAGUG
CAGUGGAAGGAAGCCACACACUGGUCAGUCAUUUCAGAGGCAGCAGAUGCCCAGGGAGACCCAA
GAAAGAGUCAGGUUAGGGAGCAGUGAAAGUGAGGAGGGAAGACAAUUCUGUGAACUCUGUAA
CUCUUAAAAUUUUUGAAAACUCCAUCGUUAAACAACUUUUAAAAGAAAUAACUAAAUUUUCAA
AUGAGUAAGCAGUGCCACCAACUAGUGUUUUGCCCGAUAGAAGAGCCAGCAUGUUCACGUUAU
UUAAAUUAGGUGGAAAAAUCUAAACAUUUUUAUCUUCAUAAUUUAAAAAAUAUAUAUGUAUA
UAUUGCAUAUUCACUUUUUCCUUUAGGUAGAGAUGAUUUCAAUCCAAAUACUCUUACUUUAA
AAAAUUUCCUUUCCCCAAGAAUCUCCUUGGGACUUUGACUUAUUUUUAAAGCUGUGUUGGAG
CUCAUCUUGUUCCCUGAUGUGUCUCGAGCCCAUUGGUAGGGUCAUACAAAGCCCACGGUUACA
AGCAGUGGUAGGAUUGCAGCCGUGGGCCUGCUGGACACACACAUACACCAAAGAUGUAUUUGG
AUCUGGGCACCCCCUCCCAGGAUCCCUGUACUCACGUGCCAGUCUCCUGACUAGAGCACUUUAC
UCUGUUUCCUCAGCCCUGCAGCCCCUGGGAGCACACACUGGGUGCAGCCCUGGGCCAGGCACGG
GAGGCCCUGCCCUGUGCUGCCCAGGGGCUGUGUGCACCACAUGAGCACAUUUCCCUCUGGCCU
GGCGGCCUCCAGGCUGGCUGUGGAAACAGUUCCUGAGGAAAUUAGAGAUUCUAUGAAUUGUA
GGAGUAUUAAAGACCAGGCUGUUGGCACCAGAACUUAAAGCGAUGACUGGAUGUCUCUGUAC
UGUAUGUAUCUGGUUAUCAAGAUGCCUCUGUGCAGAAAGUAUGCCUCCCGUGGGUAUACGUU
UUUACCUUUUUUAAAAAACAUUUUUGUAGAAAAAAUAAUUAAAUCCCCUUUUUGGAAACUUA
CUGCAGGUUUUGUGCCUUGACAACCUCUCCCUAUGUGAGGUUUGUAAAAAGUGUCCUGUGAC
UUAACACAGAAACGCAAUAAACACACACAAAAUAGUUUCAUGAGUGAUUCUUCAGAUGCCCUUC
CCAACUGGUUAGUUGAUCAAGAAUUUUGGGGGUGGGGGUUGCGGAGAAAUCAAGUUUAAAAU
UCCUUCUGAUUAAAAAAAUAUAGUGGAAUACAAUUGUCUGCCGUUUCCCCUUCUUAAUGUAU
AUAUUGUGAGUAUUUAUUAGAUUCGUAGGUCAUAUUACUUAUCAACUGAGCCAAAUGUCUGU
GUGCAAUUGUGUUUCCUUUACCUUGUAAAAUUUUGUACAGCAUAAAUAAGUAAAAAAAUCAC
UGUUUUUCUCAACUUUUUCAAAAUCAAGGAUUGUAAAUAUUGUAGAUUCUUUUUCUGUGUG
AUGUGUCCUACUGUUUCAUAAUGCUGUAACUUGUAGAAAUAUUGUAUAUUUAUUUUCUGCU
UAUUUAAUGUCUUAAUUUCUGAAAAGUAUUAACAUCCCUGUCUCCCACUCCCCUGCCGUCCCA
UGAAGUUAACUCCUGAGAGUUGUCGGGGGUGACUGGAGAGCUCAUUGCAGACCACGUGGUCC
UCCAGGGUGGCUCUCCACCUUCGGGUCCUGGUAUUUCCAGUCAAGUGGGUUUCAAUUCUUGG
GCUUUGCCGCCCUUAUGAUGAAGUGUGUGUUUGAUGCCAGUGAGAAACUCAGUCUGGCAGGC
UACAAAAUUCUACUCCAAGAAAUACCCAGCAACCUUCUGUUUGUUCCAAAGCAACUAGCUUAUC
AUGCAAGCAAAUUUUGCUGACUCCAGGCUUUAUCUUUAGGAAAACAAAAAAACCAAAGUAUUA
UCAGCAGGUGGGAAAGAUUUUUCUAUUGAAAAUUUAUCCCUGACAACUCAGCGUUUAGAAAA
GAAAUAAAAUGUGCCACUUCCAGAGGUGCUGCAUUGCAGUUGUUCAGGGCUAGGGCCAGGCAG
GACAAGUGAAUGGGUGGGACAGGUGGCUCCUGCCUAAGGACCACCUCAGGCCACUAACCCCUU
GUGGACAACUGUGAGUAGCUGGGUUUUCCCCCACCUGCUGUGCAACUUCCUGUGCUUUGAGG
UUGGACUAACUUGUCUUCAGGAGCUAAUUAACUGUACAGCCCUCCCCACGCCCCACCCAUACGG
UCACUGCAUUUGGUCAGCCUGCUUCUUCAGGUCGAUGCCCUCCUUCUGAUACUCCAUCUCCUU
CAGGGGAGGUUGGGGCCCCACUGGACUGGGUGUCAAGAUGUGAAAGCUUAUGGGAGCUUUAA
GGAGACUUCAUGGUGGUUCCAUGCAGGUGGUUCUGCCAUCCCUGCUGAUUUAGCCUGGUGCC
UGUGUGUGUCCACUCACGUACACGUGGGGUGGGGGAAACGUGUCUACAGAUGACGCUAAAUC
AGUUGGGGUCUACUCUAAACAGCAUUGUGUGUAAGAAGCAUCCUCAAGCUCCCAGUUAAGUAA
CUUGACUACUUUUAUUUGGGAAUUUCAGACUAUAGAAGCUCUCUUAUGUUUUAUGUCCAGAU
UCUGUGACCACUAGUUACUGUAUCAGAACUCAUCAGGUACCCACUUAUAAAUAGCACUGAUCU
GGCUGUAUACUGAUCCAUCACUAACCUGUUUUCUAGGACCCAGCGUAUGUAGCAUUUGUAUU
GCAGUUUCCCUGGCUUACUUGUGUUUUGCACUGAUGAAUUUUGACAGGGUAAUUGCCACUUU
ACUUGUGCAAUACUGCUGUAAAUAACUGCAGAUUUUUAAACAAUCUUUUAUGUUAAUUUUAU
AAAAAUAAAACUUUCAACUAGU

### REFERENCES

1. Kaech S M, Cui W. Transcriptional control of effector and memory CD8+ T cell differentiation. Nature reviews Immunology 2012, 12(11): 749-761.
2. Bedoui S, Heath W R, Mueller S N. CD4(+) T-cell help amplifies innate signals for primary CD8(+) T-cell immunity. Immunological reviews 2016, 272(1): 52-64.
3. Eickhoff S, Brewitz A, Gerner M Y, Klauschen F, Komander K, Hemmi H, et al. Robust Anti-viral Immunity Requires Multiple Distinct T Cell-Dendritic Cell Interactions. Cell 2015, 162(6): 1322-1337.
4. Hor J L, Whitney P G, Zaid A, Brooks A G, Heath W R, Mueller S N. Spatiotemporally Distinct Interactions with Dendritic Cell Subsets Facilitates CD4+ and CD8+ T Cell Activation to Localized Viral Infection. Immunity 2015, 43(3): 554-565.
5. Gattinoni L, Klebanoff C A, Restifo N P. Paths to stemness: building the ultimate antitumour T cell. Nature reviews Cancer 2012, 12(10): 671-684.
6. Graef P, Buchholz V R, Stemberger C, Flossdorf M, Henkel L, Schiemann M, et al. Serial transfer of single-cell-derived immunocompetence reveals stemness of CD8(+) central memory T cells. Immunity 2014, 41(1): 116-126.
7. Sukumar M, Liu J, Mehta G U, Patel S J, Roychoudhuri R, Crompton J G, et al. Mitochondrial Membrane Potential Identifies Cells with Enhanced Stemness for Cellular Therapy. Cell Metab 2016, 23(1): 63-76.
8. Shaulian E, Karin M. AP-1 as a regulator of cell life and death. Nature cell biology 2002, 4(5): E131-136.
9. Karin M, Liu Z, Zandi E. AP-1 function and regulation. Current opinion in cell biology 1997, 9(2): 240-246.
10. Murphy T L, Tussiwand R, Murphy K M. Specificity through cooperation: BATF-IRF interactions control immune-regulatory networks. Nature reviews Immunology 2013, 13(7): 499-509.
11. Li P, Spolski R, Liao W, Wang L, Murphy T L, Murphy K M, et al. BATF-JUN is critical for IRF4-mediated transcription in T cells. Nature 2012, 490(7421): 543-546.
12. lacobelli M, Wachsman W, McGuire K L. Repression of IL-2 promoter activity by the novel basic leucine zipper p21SNFT protein. Journal of immunology 2000, 165(2): 860-868.
13. Grajales-Reyes G E, Iwata A, Albring J, Wu X, Tussiwand R, Kc W, et al. Batf3 maintains autoactivation of Irf8 for commitment of a CD8alpha(+) conventional D C clonogenic progenitor. Nature immunology 2015, 16(7): 708-717.
14. Schlitzer A, Sivakamasundari V, Chen J, Sumatoh H R, Schreuder J, Lum J, et al. Identification of cDC1- and cDC2-committed DC progenitors reveals early lineage priming at the common DC progenitor stage in the bone marrow. Nature immunology 2015, 16(7): 718-728.
15. Hildner K, Edelson B T, Purtha W E, Diamond M, Matsushita H, Kohyama M, et al. Batf3 deficiency reveals a critical role for CD8alpha+ dendritic cells in cytotoxic T cell immunity. Science 2008, 322(5904): 1097-1100.
16. Edelson B T, Kc W, Juang R, Kohyama M, Benoit L A, Klekotka P A, et al. Peripheral CD103⁺ dendritic cells form a unified subset developmentally related to CD8alpha+conventional dendritic cells. The Journal of experimental medicine 2010, 207(4): 823-836.
17. Tussiwand R, Lee W L, Murphy T L, Mashayekhi M, Kc W, Albring J C, et al. Compensatory dendritic cell development mediated by BATF-IRF interactions. Nature 2012, 490(7421): 502-507.
18. Betz B C, Jordan-Williams K L, Wang C, Kang S G, Liao J, Logan M R, et al. Batf coordinates multiple aspects of B and T cell function required for normal antibody responses. The Journal of experimental medicine 2010, 207(5): 933-942.
19. Karwacz K, Miraldi E R, Pokrovskii M, Madi A, Yosef N, Wortman I, et al. Critical role of IRF1 and BATF in forming chromatin landscape during type 1 regulatory cell differentiation. Nature immunology 2017, 18(4): 412-421.
20. Punkenburg E, Vogler T, Buttner M, Amann K, Waldner M, Atreya R, et al. Batf-dependent Th17 cells critically regulate IL-23 driven colitis-associated colon cancer. Gut 2016, 65(7): 1139-1150.
21. Schraml B U, Hildner K, Ise W, Lee W L, Smith W A, Solomon B, et al. The AP-1 transcription factor Batf controls T(H)17 differentiation. Nature 2009, 460(7253): 405-409.
22. Kuroda S, Yamazaki M, Abe M, Sakimura K, Takayanagi H, Iwai Y. Basic leucine zipper transcription factor, ATF-like (BATF) regulates epigenetically and energetically effector CD8 T-cell differentiation via Sirt1 expression. Proceedings of the National Academy of Sciences of the U.S. Pat. No. 2,011,108(36): 14885-14889.
23. Kurachi M, Barnitz R A, Yosef N, Odorizzi P M, Dilorio M A, Lemieux M E, et al. The transcription factor BATF operates as an essential differentiation checkpoint in early effector CD8+ T cells. Nature immunology 2014, 15(4): 373-383.
24. Quigley M, Pereyra F, Nilsson B, Porichis F, Fonseca C, Eichbaum Q et al. Transcriptional analysis of HIV-specific CD8+ T cells shows that PD-1 inhibits T cell function by upregulating BATF. Nature medicine 2010, 16(10): 1147-1151.
25. Logan M R, Jordan-Williams K L, Poston S, Liao J, Taparowsky E J. Overexpression of Batf induces an apoptotic defect and an associated lymphoproliferative disorder in mice. Cell death & disease 2012, 3: e310.
26. Man K, Gabriel S S, Liao Y, Gloury R, Preston S, Henstridge D C, et al. Transcription Factor IRF4 Promotes CD8(+) T Cell Exhaustion and Limits the Development of Memory-like T Cells during Chronic Infection. Immunity 2017, 47(6): 1129-1141 e1125.
27. Gerlach C, Moseman E A, Loughhead S M, Alvarez D, Zwijnenburg A J, Waanders L, et al. The Chemokine Receptor CX3CR1 Defines Three Antigen-Experienced CD8 T Cell Subsets with Distinct Roles in Immune Surveillance and Homeostasis. Immunity 2016, 45(6): 1270-1284.
28. Bottcher J P, Beyer M, Meissner F, Abdullah Z, Sander J, Hochst B, et al. Functional classification of memory CD8(+) T cells by CX3CR1 expression. Nature communications 2015, 6: 8306.
29. van der Windt G J, Everts B, Chang C H, Curtis J D, Freitas T C, Amiel E, et al. Mitochondrial respiratory capacity is a critical regulator of CD8⁺ T cell memory development. Immunity 2012, 36(1): 68-78.
30. Pearce E L, Walsh M C, Cejas P J, Harms G M, Shen H, Wang L S, et al. Enhancing CD8 T-cell memory by modulating fatty acid metabolism. Nature 2009, 460(7251): 103-10⁷.
31. O'Neill L A, Kishton R J, Rathmell J. A guide to immunometabolism for immunologists. Nature reviews Immunology 2016, 16(9): 553-565.
32. Yamazaki C, Sugiyama M, Ohta T, Hemmi H, Hamada E, Sasaki I, et al. Critical roles of a dendritic cell subset expressing a chemokine receptor, XCR1. Journal of immunology 2013, 190(12): 6071-6082.
33. Alexandre Y O, Ghilas S, Sanchez C, Le Bon A, Crozat K, Dalod M. XCR1⁺ dendritic cells promote memory CD8⁺ T cell recall upon secondary infections with Listeria monocytogenes or certain viruses. The Journal of experimental medicine 2016, 213(1): 75-92.
34. Mattiuz R, Wohn C, Ghilas S, Ambrosini M, Alexandre Y O, Sanchez C, et al. Novel Cre-Expressing Mouse Strains Permitting to Selectively Track and Edit Type 1 Conventional Dendritic Cells Facilitate Disentangling Their Complexity in vivo. Frontiers in immunology 2018, 9: 2805.
35. Kitano M, Yamazaki C, Takumi A, lkeno T, Hemmi H, Takahashi N, et al. Imaging of the cross-presenting dendritic cell subsets in the skin-draining lymph node. Proceedings of the National Academy of Sciences of the U.S. Pat. No. 2,016,113(4): 1044-1049.
36. D'Cruz L M, Rubinstein M P, Goldrath A W. Surviving the crash: transitioning from effector to memory CD8⁺ T cell. Seminars in immunology 2009, 21(2): 92-98.
37. Pellegrini M, Belz G, Bouillet P, Strasser A. Shutdown of an acute T cell immune response to viral infection is mediated by the proapoptotic Bcl-2 homology 3-only protein Bim. Proceedings of the National Academy of Sciences of the United States of America 2003, 100(24): 14175-14180.
38. Hildeman D A, Zhu Y, Mitchell T C, Bouillet P, Strasser A, Kappler J, et al. Activated T cell death in vivo mediated by proapoptotic bcl-2 family member bim. Immunity 2002, 16(6): 759-767.
39. Sionov R V, Vlahopoulos S A, Granot Z. Regulation of Bim in Health and Disease. Oncotarget 2015, 6(27): 23058-23134.
40. Hajji N, Joseph B. Epigenetic regulation of cell life and death decisions and deregulation in cancer. Essays in biochemistry 2010, 48(1): 121-146.
41. Shaulian E, Karin M. AP-1 in cell proliferation and survival. Oncogene 2001, 20(19): 2390-2400.
42. Borst J, Ahrends T, Babala N, Melief CJM, Kastenmuller W. CD4(+) T cell help in cancer immunology and immunotherapy. Nature reviews Immunology 2018, 18(10): 635-647.
43. Miyagawa F, Zhang H, Terunuma A, Ozato K, Tagaya Y, Katz S I. Interferon regulatory factor 8 integrates T-cell receptor and cytokine-signaling pathways and drives effector differentiation of CD8 T cells. Proceedings of the National Academy of Sciences of the U.S. Pat. No. 2,012,109(30): 12123-12128.
44. Wei J, Long L, Zheng W, Dhungana Y, Lim S A, Guy C, et al. Targeting REGNASE-1 programs long-lived effector T cells for cancer therapy. Nature 2019, 576(7787): 471-476.
45. Lynn R C, Weber E W, Sotillo E, Gennert D, Xu P, Good Z, et al. c-Jun overexpression in CAR T cells induces exhaustion resistance. Nature 2019, 576(7786): 293-300.
46. Lopez-Bergami P, Lau E, Ronai Z. Emerging roles of ATF2 and the dynamic AP1 network in cancer. Nature reviews Cancer 2010, 10(1): 65-76.
47. Weiser C, Petkova M V, Rengstl B, Doring C, von Laer D, Hartmann S, et al. Ectopic expression of transcription factor BATF3 induces B-cell lymphomas in a murine B-cell transplantation model. Oncotarget 2018, 9(22): 15942-15951.
48. Dobin A, Davis C A, Schlesinger F, Drenkow J, Zaleski C, Jha S, et al. STAR: ultrafast universal RNA-seq aligner. Bioinformatics 2013, 29(1): 15-21.
49. Liao Y, Smyth G K, Shi W. The R package Rsubread is easier, faster, cheaper and better for alignment and quantification of RNA sequencing reads. Nucleic acids research 2019, 47(8): e47.
50. Love M I, Huber W, Anders S. Moderated estimation of fold change and dispersion for RNA-seq data with DESeq2. Genome biology 2014, 15(12): 550.
51. Agarwal S, Weidner T, Thalheimer F, Buchholz C. In vivo generated human CAR T cells eradicate tumor cells. Oncoimmunology. 2019; 8(12):e1671761.
52. Agarwal S, Hanauer JDS, Frank A M, Riechert V, Thalheimer F B, Buchholz C J. In vivo generation of CART cells selectively in human CD4⁺ lymphocytes. Mol Ther. 28(8):1783-1794.
53. Smith T T, Stephan S B, Moffett H F, McKnight L E, Ji W, Reiman D, Bonagofski E, Wohlfahrt M E, Pillai SPS, Stephan M T. In situ programming of leukaemia-specific T cells using synthetic DNA nanocarriers. Nat Nanotechnol. 2017 August; 12(8):813-820.

1. Lymphocyte, modified to exhibit a reduced Bcl-2-like protein 11 (BCL2L11) level and/or increased BATF3 level.

2. Lymphocyte according to claim 1 that is modified to exhibit a reduced BCL2L11 level.

3. Lymphocyte according to any one of the preceding claims, wherein the BCL2L11 level is a BCL2L11 protein activity level and/or a BCL2L11 protein quantity level, preferably a BCL2L11 protein quantity level.

4. Lymphocyte according to any one of the preceding claims, comprising an exogenous inhibitor of BCL2L11 protein activity in the lymphocyte.

5. Lymphocyte according to any one of the preceding claims, comprising an exogenous activator of BCL2L11 protein degradation in the lymphocyte.

6. Lymphocyte according to any one of the preceding claims, wherein the BCL2L11 level is a BCL2L11 expression level.

7. Lymphocyte according to any one of the preceding claims, comprising an exogenous inhibitor of BCL2L11 expression in the lymphocyte.

8. Lymphocyte according to any one of the preceding claims, comprising an exogenous inhibitor of transcription from an endogenous BCL2L11 gene and/or an inhibitor of translation of an endogenous BCL2L11 mRNA.

9. Lymphocyte according to any one of the preceding claims, comprising an exogenous RNAi effector molecule for reducing BCL2L11 expression in the lymphocyte.

10. Lymphocyte according to claim 9, wherein the RNAi effector molecule is an siRNA or an shRNA for reducing BCL2L11 expression in the lymphocyte.

11. Lymphocyte according to any one of the preceding claims, comprising an expression cassette for an siRNA or an shRNA for reducing BCL2L11 expression in the lymphocyte, wherein the expression cassette optionally has been integrated into the genome of lymphocyte.

12. Lymphocyte according to any one of the preceding claims, in which an endogenous BCL2L11 gene has been genetically modified in order to reduce or disrupt BCL2L11 expression, wherein optionally the endogenous BCL2L11 gene has been genetically modified in its promoter region.

13. Lymphocyte according to any one of the preceding claims that is modified to exhibit an increased BATF3 level, preferably human or murine BATF3 level.

14. Lymphocyte according to any one of the preceding claims, wherein the BATF3 level is a BATF3 protein activity level and/or a BATF3 protein quantity level, preferably a BATF3 protein quantity level.

15. Lymphocyte according to any one of the preceding claims, comprising an activator of BATF3 protein activity in the lymphocyte.

16. Lymphocyte according to any one of the preceding claims, comprising an exogenous inhibitor of BATF3 protein degradation in the lymphocyte.

17. Lymphocyte according to any one of the preceding claims, wherein the BATF3 level is a BATF3 expression level in the lymphocyte.

18. Lymphocyte according to any one of the preceding claims, comprising an exogenous activator of BATF3 expression in the lymphocyte.

19. Lymphocyte according to any one of the preceding claims, comprising an exogenous nucleic acid molecule for overexpressing BATF3, optionally human BATF3, wherein the nucleic acid molecule optionally has been integrated into the genome of the lymphocyte.

20. Lymphocyte according to any one of the preceding claims, comprising an exogenous activator of transcription from an endogenous BATF3 gene and/or activator of translation of an endogenous BATF3 mRNA.

21. Lymphocyte according to any one of the preceding claims, in which an endogenous BATF3 gene has been genetically modified in order to increase BATF3 expression, wherein optionally the endogenous BATF3 gene has been genetically modified in its promoter region.

22. Lymphocyte,
a) comprising an exogenous inhibitor of BCL2L11 protein activity in the lymphocyte;
or
b) comprising an exogenous activator of BCL2L11 protein degradation; or
c) comprising an exogenous inhibitor of BCL2L11 expression in the lymphocyte and/or an (exogenous) expression cassette for expressing an inhibitor of BCL2L11 expression in the lymphocyte;
or
d) in which an endogenous BCL2L11 gene has been genetically modified in order to reduce or disrupt BCL2L11 expression, wherein optionally the endogenous BCL2L11 gene has been genetically modified in its promoter region.

23. Lymphocyte,
a) comprising an exogenous activator of BATF3 protein activity in the lymphocyte; or
b) comprising an exogenous inhibitor of BATF3 protein degradation; or
c) comprising an exogenous activator of BATF3 expression in the lymphocyte and/or a (exogenous) nucleic acid molecule for overexpressing BATF3 in the lymphocyte; or
d) in which an endogenous BATF3 gene has been genetically modified in order to increase BATF3 expression, wherein optionally the endogenous BATF3 gene has been genetically modified in its promoter region.

24. Method for producing a lymphocyte that exhibits a reduced BCL2L11 level and/or increased BATF3 level, wherein the method comprises
a) introducing into the lymphocyte an exogenous inhibitor of BCL2L11 protein activity in the lymphocyte; or
b) introducing into the lymphocyte an exogenous activator of BCL2L11 protein degradation in the lymphocyte; or
c) introducing into the lymphocyte an exogenous inhibitor of BCL2L11 expression in the lymphocyte or an expression cassette for expressing an inhibitor of BCL2L11 expression in the lymphocyte; or
d) genetically modifying an endogenous BCL2L11 gene in order to reduce or disrupt BCL2L11 expression, wherein optionally the endogenous BCL2L11 gene has been genetically modified in its promoter region; or
e) introducing into the lymphocyte an exogenous activator of BATF3 protein activity in the lymphocyte; or
f) introducing into the lymphocyte an exogenous inhibitor of BATF3 protein degradation; or
g) introducing into the lymphocyte an exogenous activator of BATF3 expression and/or a (exogenous) nucleic acid molecule for overexpressing BATF3 in the lymphocyte; or
h) genetically modifying an endogenous BATF3 gene in the lymphocyte in order to increase BATF3 expression, wherein optionally the endogenous BATF3 gene has been genetically modified in its promoter region.

25. Lymphocyte obtainable by the method according to claim 24.

26. Lymphocyte according to any one of claim 1-23 or 25, or method according to claim 24, wherein the lymphocyte is a mammalian lymphocyte.

27. Lymphocyte according to any one of claim 1-23 or 25, or method according to claim 24, wherein the lymphocyte is a human lymphocyte.

28. Lymphocyte according to any one of claim 1-23 or 25, or method according to claim 24, or lymphocyte or method according to any one of claims 26-27, wherein the lymphocyte is a T cell or an innate lymphoid cell, such as an ILC1, ILC2, ILC3 or NK cell.

29. Lymphocyte according to any one of claim 1-23 or 25, or method according to claim 24, or lymphocyte or method according to any one of claims 26-28, wherein the lymphocyte is a T cell, such as a CD8+ T cell or a CD4+ T cell.

30. Lymphocyte according to any one of claim 1-23 or 25, or method according to claim 24, or lymphocyte or method according to any one of claims 26-29, wherein the lymphocyte is a CD8+ T cell.

31. Lymphocyte according to any one of claim 1-23 or 25, or method according to claim 24, or lymphocyte or method according to any one of claims 26-30, wherein the lymphocyte is a cell expressing a chimeric antigen receptor (CAR).

32. Lymphocyte according to any one of claim 1-23 or 25, or method according to claim 24, or lymphocyte or method according to any one of claims 26-31, wherein the lymphocyte is a T cell expressing a chimeric antigen receptor (CAR).

33. Lymphocyte according to any one of claim 1-23 or 25, or method according to claim 24, or lymphocyte or method according to any one of claims 26-32, wherein the lymphocyte exhibits increased survival in a (human) subject, for example after administration to the subject.

34. Lymphocyte according to any one of claim 1-23 or 25, or method according to claim 24, or lymphocyte or method according to any one of claims 26-33, wherein the lymphocyte exhibits increased expression of IL7R in a (human) subject, for example after administration to the subject.

35. Composition comprising a lymphocyte according to any one of claim 1-23 or 25-34, wherein the composition is optionally a pharmaceutical composition.

36. Lymphocyte according to any one of claim 1-23 or 25-34, or composition according to claim 35 for use in medicine.

37. Lymphocyte according to any one of claim 1-23 or 25-34, or composition according to claim 35 for use in treating cancer, an infectious disease, or an autoimmune disease, such as a chronic inflammatory disease or degenerative disease.

38. Lymphocyte for use according to claim 36 or 37, wherein the lymphocyte exhibits an improved therapeutic activity and/or therapeutic outcome.
